(19) United States
(12) Patent Application Publication
(10) Pub. No.: US 2015/0368195 A1
(43) Pub. Date: Dec. 24, 2015

(54) INDOLINE COMPOUNDS FOR TREATMENT AND/OR PREVENTION OF INFLAMMATION DISEASES

(71) Applicant: Taipei Medical University, Taipei (TW)

(72) Inventors: Jing-Ping Liou, TAIPEI (TW); Chien-Huang Lin, TAIPEI (TW); Shiow-Lin Pan, TAIPEI (TW); Chia-Ron Yang, TAIPEI (TW); Che-Ming Teng, TAIPEI (TW)

(21) Appl. No.: 14/310,587

(22) Filed: Jun. 20, 2014

(51) Int. Cl. C07D 209/08

(52) U.S. Cl. CPC C07D 209/08

(57) ABSTRACT

The invention is based on the discovery that 1-arylsulfonyl-5-(N-hydroxyacrylamide)indolines and 1-arylsulfonyl-5-(N-hydroxyacrylamide)indoles has great potential as a novel agent to be used in the treatment of inflammation-associated diseases, particularly, inflammatory arthritis and fibrosis.

### FIELD OF THE INVENTION

The invention relates to a method for treating and/or preventing an inflammation disease comprising administering indoline compounds. Particularly, the method uses 1-arylsulfonyl-5-(N-hydroxyacrylamide)indolines or 1-arylsulfonyl-5-(N-hydroxyacrylamide)indoles to treat and/or prevent inflammation diseases.

### BACKGROUND OF THE INVENTION

Cytokines are soluble proteinaceous substances produced by a wide variety of haemopoietic and non-haemopoietic cell types, and are critical to the functioning of both innate and adaptive immune responses. Apart from their role in the development and functioning of the immune system, and their aberrant modes of secretion in a variety of immunological, inflammatory and infectious diseases, cytokines are also involved in several developmental processes during human embryogenesis. Thus, cytokines often act locally, but can also have effects on the whole body. For example, cytokines are able to interact directly with the evolving biology of an injury, trauma, or disease. Compounds having cytokine mediating activity have application in rheumatoid arthritis and inflammation.

Inflammatory events play a central role in the pathology of disease conditions and this process is mediated by cytokines, a system of polypeptides that enable one cell to signal to initiate events in another cell that initiate inflammatory sequelae. Normally, the system acts as part of a defensive reaction against infectious agents, harmful environmental agents, or malignantly transformed cells. But when inflammation exceeds the requirements of its defensive role, it can initiate adverse clinical effects, such as arthritis, septic shock, inflammatory bowel disease, and a range of other human disease conditions.

As one example, fibrosis of organs occurs in such a manner that extracellular matrix is excessively accumulated in the organs through invasion or injury of organs due to some cause. Excessive deposition of extra cellular matrix (ECM) components such as fibronectin (FN) and type I collagen by organ fibroblasts is defined as fibrosis. Organ fibrosis is the final common pathway for many diseases that result in end-stage organ failure. Uncontrollable wound-healing responses, including acute and chronic inflammation, angiogenesis, activation of resident cells, and ECM remodeling, are thought to be involved in the pathogenesis of fibrosis. However, effective therapy for organ fibrosis is still unavailable

As another example, rheumatoid arthritis (RA) is a systemic chronic autoimmune disease that results in destructive arthropathy. The complex interactions between the synovial and immune system cells result in synoviocyte proliferation, release of inflammatory cytokines/chemokines that recruit immune cells into the affected joints and activate infiltrated cells, and expression of degradative enzymes, resulting in progressive joint damage. Thus, these two cell types are key effector cells in RA and provide targets for pathological investigation and drug development. Classic drugs used for treating RA fall into three categories: nonsteroidal anti-inflammatory drugs (NSAIDs), steroids, and disease-modifying anti-rheumatic drugs (DMARDs). However, some adverse effects of these drugs remain major concerns. Recently developed therapies that targeted cytokines have a major impact on the disease course of RA, however, its usage may be difficult because of increased risks of infection and nonresponse rates. Therefore, novel treatments that target critical intracellular molecules in synovial inflammation are required.

Histone deacetylases (HDACs) are categorized into four categories: class I (HDAC1, 2, 3, and 8); class IIa (HDAC4, 5, 7, and 9) and class IIb (HDAC6 and 10); class III (SIRT1-7); and class IV (HDAC11). These are involved in the post-translational modifications of core histone and nonhistone proteins. Recent proteomic analyses have shown that a substantial number of key signal transduction components and transcription factors that regulate immune responses and inflammation are HDAC substrates (Choo Q Y, Ho P C, Lin H S. Histone deacetylase inhibitors: new hope for rheumatoid arthritis? Curr Pharm Des 2008; 14: 803-820; Shakespear M R, Halili M A, Irvine K M, Fairlie D P, Sweet M J. Histone deacetylases as regulators of inflammation and immunity. Trends Immunol 2011; 32: 335-343). Thus, HDAC inhibitors have been examined as possible anti-inflammatory agents. However, there are very few HDAC inhibitors that have been sufficiently developed to undergo clinical trials for RA treatment.

ITF2357 (givinostat) ameliorated joint inflammation and prevented cartilage and bone destruction in an animal model (Joosten L A, Leoni F, Meghji S, Mascagni P. Inhibition of HDAC activity by ITF2357 ameliorates joint inflammation and prevents cartilage and bone destruction in experimental arthritis. Mol Med 2011; 17: 391-396). However, a phase II safety and efficacy clinical trial of ITF2357 that evaluated patients with active systemic onset of juvenile idiopathic arthritis, but not those with RA, suggested that HDAC inhibitors still require considerable development for use as RA therapeutics.

Therefore, there is still a need to develop an anti-inflammatory candidate; particularly, a candidate against RA and fibrosis.

### SUMMARY OF THE INVENTION

The invention provides a method for inhibiting cytokine release from a cell, comprising administering an effective amount of the compound having formula (I) or a pharmaceutically acceptable salt, prodrug or solvate thereof to a subject:

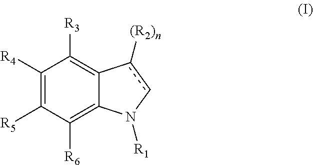

wherein [symbol] is a single bond or a double bond;

R₁ is SO₂Rₐ, wherein Rₐ is a aryl unsubstituted or substituted by 1 to 3 substituent selected from the group consisting of: —OC₁₋₁₀alkyl, halogen, —NO₂, —NH₂, —OH, —C₁₋₆alkyl, —C₂₋₁₀alkenyl, —C₂₋₁₀alkynyl, —C₃₋₁₀cycloalkyl, —C₅₋₁₀cycloalkenyl, 6 to 10 membered aryl or 6 to 10 membered heteroaryl;

R₂, R₅ and R₆ are each independently H, —OC₁₋₁₀alkyl, halogen, —NO₂, —NH₂, —OH, —C₁₋₁₀alkyl, —C₂₋₁₀alkenyl or —C₂₋₁₀alkynyl; and

R₄ is H, C₁₋₁₀alkyl, C₂₋₁₀alkenyl, C₂₋₁₀alkynyl, aryl, 5 to 14 membered heteroaryl, C₃₋₁₀cycloalkyl, C₅₋₁₀cycloalkenyl, C₅₋₁₄heterocycloC₁₋₁₀alkyl, C₅₋₁₄heterocyclo C₂₋₁₀alkenyl, halo, cyano, nitro, OR_{b}, SR_{b}, S(O)R_{b}, CH═CH—C(O)NR_{c}R_{d}, NHC(O)—CH═CH—C(O)R_{b}, NHC(O)—CH═CH—C(O)NRcRd, SO₂NRcRd, OC(O)R_{b}, C(O)NR_{c}R_{d}, NRcRd, NHC(O)R_{b}, NHC(O)NR_{c}R_{d}, or NHC(S)Rc, in which each of R_{b}, R_{c}, and R_{d}, independently, is H, hydroxy, C₁₋₁₀alkoxy, C₆₋₁₀aryloxy, C₅₋₁₄heteroaryloxy, C₁₋₁₀alkyl, C₂₋₁₀alkenyl, C₂₋₁₀alkynyl, C₆₋₁₀aryl, C₅₋₁₄heteroaryl, C₃₋₁₀cycloalkyl, C₅₋₁₀cycloalkenyl, C₃₋₁₄heterocycloC₁₋₆alkyl, or C₅₋₁₄heterocycloC₂₋₁₀alkenyl.

the invention provides a method for inhibiting HDACs 1, 2, 3, and 8 in a cell or a subject, comprising administering an effective amount of the compound having formula (I) or a pharmaceutically acceptable salt, prodrug or solvate thereof to the cell or subject.

In one embodiment, the inhibition of cytokine release is associated with an inflammatory disease, particularly, a chronic inflammation disease. The inflammatory disease include, but not limited to, arthritis, synovitis, vasculitis, conditions associated with inflammation of the bowel, atherosclerosis, multiple sclerosis, Alzheimer's disease, vascular dementia, pulmonary inflammatory diseases, fibrotic diseases, inflammatory diseases of the skin, systemic inflammatory response syndrome, sepsis, inflammatory and/or an autoimmune disorder (for example, autoimmune conditions of the liver, and/or the complications thereof. Preferably, the arthritis is osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease) or Still's disease. Preferably, the fibrosis is pulmonary fibrosis, liver fibrosis or renal fibrosis.

### BRIEF DESCRIPTION OF THE DRAWING

FIGS. 1A-H show that MPT0G009 inhibits inflammatory mediator production and cell proliferation. (FIG. 1A) Structure of MPT0G009. (FIG. 1B) RAW264.7 cells (1×10⁶) and (FIG. 1C) RA-FLS (2.5×10⁴) were incubated for 30 min with or without MPT0G009 (0.1, 1, or 10 μM) or suberoylanilide hydroxamic acid (SAHA; 0.3, 3, or 30 μM). Then either lipopoysaccharide (LPS, 25 ng/mL) was added for 24 h and culture supernatants were assayed for prostaglandin E2 (PGE₂) and nitrites or interleukin (IL)-1β (10 ng/mL) was added for 24 h and IL-6 levels were measured. (FIG. 1D) HIG-82 synoviocytes and (FIG. 1E) RA-FLS (5×10³) were incubated for 48 h with or without MPT0G009 or SAHA, and their anti-proliferative effects were determined by an sulforhodamine B (SRB) assay. (FIGS. 1F and G) RA-FLS (1×10⁶) were incubated for 24 h with or without MPT0G009 or SAHA, fixed, and then stained with propidium iodide to analyze (FIG. 1F) the DNA contents by flow cytometry and (FIG. 1G) cell cycle distributions. (FIG. 1H) RA-FLS (1×10⁶) were incubated for 24 h with or without MPT0G009 (1 μM) or SAHA (3 μM) and then with an anti-p21 antibody to determine the expression of p21 by flow cytometry. Results in (FIGS. 1B-E) and (FIG. 1G) are means±SEM's for three independent experiments, *p<0.05 and **p<0.01 compared with no added inhibitor.

FIGS. 2A-C show the effects of MPT0G009 (1 or 10 μM) and SAHA (3 or 30 μM) at the above mentioned concentrations on the proliferation of HIG-82 synoviocytes or RA-FLS after 24 or 48 h of incubation (FIGS. 2A and B) and the effect on cyclin-dependent kinase inhibitors, such as p21, by incubating RA-FLS with 1 μM MPT0G009 or 3 μM SAHA for 24 h and assessed the expression of p21 by western blot (FIG. 2C).

FIGS. 3A-B shows the concentration-dependently inhibitory effects of Compound 9 and SAHA (1) on the LPS-induced protein levels of inducible nitric oxide synthase (iNOS) and cyclooxygenase-2 (COX-2). RAW264.7 macrophages (1×10⁶) in 6-well plates were incubated with 0.1-10 μM Compound 9 (FIG. 3A) or SAHA (FIG. 3B) for 30 min, followed by stimulation with LPS (25 ng/mL) for 24 h. Then the cells were harvested and whole cell extracts were prepared for Western blot analysis for iNOS and COX-2. The relative protein expressions are presented as mean±SEM for four replicates. *p<0.05 compared to the LPS-treated group.

FIGS. 4A-B shows that compound 9 and SAHA (1) suppress carrageenan-induced hind paw edema in rats. A 0.5% (w/v) suspension of carrageenan in normal saline was administered to male Wistar rats (7-weeks) by intradermal injection into the base of the right hind paw. One hour prior to carrageenan injection, rats were oral administration of vehicle or a fine suspension of Compound 9 (25 mg/kg), SAHA (200 mg/kg) in vehicle. A positive control group was included in which rats were pretreated with 5 mg/kg Indomethacin. Three hours after carrageenan administration, the thickness (FIG. 4A) and volume (FIG. 4B) of the right hind paw were measured by digital caliper and digital plethysmometer, respectively. The results are expressed as the mean±SEM, with n=5. *p<0.05 compared to the control group; #p<0.05 for the comparison of the indicated groups.

FIGS. 5A-C show that MPT0G009 increases the expression of acetyl histone 3 proteins in synovial fibroblasts. (FIG. 5A) HIG-82 cells and (FIG. 5B) RA-FLS (1×10⁶) in 6-well plates were either untreated for 24 h or were incubated for the indicated times with MPT0G009 (3 μM for HIG-82 cells; 1 μM for RA-FLS) or suberoylanilide hydroxamic acid (SAHA; 60 μM for HIG-82 cells; 30 μM for RA-FLS). Then cells were harvested and cell lysates were prepared for Western blot analysis of the indicated proteins. (FIG. 5C) RA-FLS were incubated for 1 h with or without the proteasome inhibitor, MG132 (1 μM). Cells were then incubated with or without MPT0G009 (1 μM) or SAHA (30 μM) for another 24 h in the continued presence or absence of the inhibitor, after which cell lysates were prepared for Western blot analysis of the histone deacetylase inhibitor (HDAC3). Results shown are representative of three independent experiments. The numbers below each blot are the mean quantitative results as measured by densitometry relative to that without MPT0G009 or SAHA.

FIGS. 6A-E show that MPT0G009 inhibits the formation of osteoclast-like multinuclear cells by RAW264.7 macrophages. (FIG. 6A) RAW264.7 cells (1×10³) were incubated for 30 min with MPT0G009 (5 nM) or suberoylanilide hydroxamic acid (SAHA; 50 nM), after which macrophage colony-stimulating factor (M-CSF) and receptor activator of NF-κB Ligand (RANKL; 50 ng/mL each) were added, and incubation was continued for 5 days. Cells were then tartrate resistant acid phosphatase (TRAP) stained, and the numbers of TRAP-positive multinuclear cells were counted. (FIG. 6B) RAW264.7 cells were incubated with 5 nM MPT0G009 or 50 nM SAHA and treated as in (FIG. 6A), after which their morphology was examined by light microscopy. Images are at ×100 magnification, and the arrows indicate differentiated osteoclasts. (FIG. 6C) RAW264.7 cells were treated as in (FIG. 6B), then incubated with a fluorescein isothiocyanate (FITC)-conjugated anti-CD51/61 antibody and analyzed by flow cytometry. (FIGS. 6D and 6E) RAW264.7 cells (1×10⁵) were transfected with 1 μg of pGL4.32[luc2P/NF-κB-RE/Hygro] (FIG. 6D) or pGL4.30[luc2P/NFAT-RE/Hygro] (FIG. 6E) for 24 h and then incubated for 30 min with or without MPT0G009 (5 nM). Then RANKL (50 ng/mL) was added and incubation was continued for an additional 24 h, after which luciferase activity was determined. Results in (FIGS. 6A, 6D, and 6E) are the means±SEM's for three independent experiments. **p<0.01 compared with the indicated controls.

FIGS. 7A-E shows that overexpression of histone deacetylase 1 (HDAC1) and HDAC6 reduces MPT0G009 inhibition of cytokine secretion and osteoclast differentiation. (FIG. 7A) RAW264.7 macrophages or RA-FLS (1×10⁶) were transfected for 24 h with 1 μg of an empty vector or a vector encoding for HDAC1-Flag and/or HDAC6-Flag, after which cell lysates were immunoprecipitated with 1 μg of an anti-Flag antibody and immunoblotted for the indicated proteins. (FIG. 7B) RAW264.7 cells (1×10⁶) and (FIG. 7C) RA-FLS (2.5×10⁴) that were transfected with an empty vector or vectors encoding for both HDAC1 and HDAC6 as in FIG. 7A were incubated for 30 min with or without 10 μM of MPT0G009 or 30 μM of suberoylanilide hydroxamic acid (SAHA). Then lipopolysaccharide (LPS; 25 ng/mL) was added for another 24 h, and culture supernatants were assayed for nitric oxide or prostaglandin E2 (PGE₂). (FIGS. 7D and E) RAW264.7 cells (1×10³) transfected as in FIG. 7B were incubated for 30 min with or without 5 nM of MPT0G009 or 50 nM of SAHA, after which macrophage colony-stimulating factor (M-CSF) and receptor activator of NF-κB Ligand (RANKL; 50 ng/mL each) were added and incubated for 5 days. Cells were then tartrate resistant acid phosphatase

(TRAP) stained, and the numbers of TRAP-positive multinuclear cells were counted (FIG. 7D) or were incubated with a fluorescein isothiocyanate (FITC)-conjugated anti-CD51/61 antibody and analyzed by flow cytometry (FIG. 7E). Results in FIGS. 7B-D are means±SEM's for three independent experiments. *p<0.05.

FIGS. 8A-F show that MPT0G009 inhibits the development of arthritis in an adjuvant-induced arthritis (AIA) model. (FIG. 8A) After the onset of arthritis (as described in the Materials and Methods section), rats were orally treated with either the vehicle (MPT0G009; 25 mg/kg), suberoylanilide hydroxamic acid (SAHA; 200 mg/kg), or the positive control indomethacin (1 mg/kg) from days 2 to 21 (19 days). Subsequently, swelling of both hind paws was photographed. (FIG. 8B) Hind paw volumes in the indicated group of rats were measured using a digital plethysmometer on the indicated day after AIA induction. (FIG. 8C) Arthritis scores on day 21. (FIG. 8D) Serum levels of interleukin (IL)-1β (left panel) and IL-6 (right panel) on day 21 as measured by enzyme-linked immunosorbent assay (ELISA). (FIG. 8E) Top five rows: Photomicrographs of ankle joint sections from the different groups stained with hematoxylin and eosin, safranin O, immunohistochemically stained with an anti-acetyl H3 antibody or TRAP stain. Arrows indicate osteoblasts (OB); arrowheads indicate osteoclasts (OC). Bottom panels: Micro-computed tomography results (arrows indicate bone erosion). (FIG. 8F) The bone mineral density (BMD, in mg/mm³) and the bone mineral content (BMC, in mg) values for the bone tissue of the tarsus were analyzed using CT Analysis Software. Results in (FIGS. 8B-D and F) are the means±SEM's for five independent experiments. *p<0.05 compared with the corresponding day 0 value (FIG. 8B); # p<0.05 compared with the vehicle-treated control (FIGS. 8B-D); **p<0.01 and ***p<0.001 compared with basal groups (FIG. 8F).

FIG. 9 shows that MPT0G009 suppresses fibroblast-like synoviocytes proliferation and inflammation in the adjuvant-induced arthritis (AIA) model. Rats were orally treated with the vehicle, MPT0G009 (25 mg/kg), or suberoylanilide hydroxamic acid (SAHA; 200 mg/kg) after the onset of arthritis from day 2 to 21 (19 days). Subsequently, photomicrographs of ankle joint sections from the different groups by immunohistochemically staining with KI-67 (upper panel; images represent magnification at 100×) or COX-2 (lower panel; images represent magnification at 200×) antibodies. Scale bar=100 and 50 μM respectively.

FIGS. 10A-E shows that MPT0E028 and MPT0G009 inhibit pro-fibrogenic mediators-induced fibrotic protein, CTGF and collagen I, production. Western blot analysis was performed. First, WI-38 lung fibroblasts were incubated with different concentrations of MPT0E028 (0.01, 0.03, 0.1, 0.3, or 1 μM) (FIG. 10A) or MPT0G009 (0.01, 0.03, 0.1, 0.3, or 1 μM) (FIG. 10B) for 30 min before and during incubation for 2 h with TGF-β (10 ng/mL). WI-38 lung fibroblasts were incubated with 1 μM MPT0E028 and MPT0G009 for 30 min and following another incubation for 2 h with 1 U/ml thrombin (FIG. 10C) and 10 nM ET-1 (FIG. 10D). The collagen I production from WI-38 lung fibroblasts stimulated by 10 ng/mL TGF-β for 24 h was inhibited with 1 μM MPT0E028 and MPT0G009 pretreatment (FIG. 10E).

### DETAILED DESCRIPTION OF THE INVENTION

The invention is, at least in part, based on the discovery that 1-arylsulfonyl-5-(N-hydroxyacrylamide)indolines and 1-arylsulfonyl-5-(N-hydroxyacrylamide)indoles has great potential as a novel agent to be used in the treatment of inflammation-associated diseases, particularly, inflammatory arthritis and fibrosis.

The invention surprisingly found that the compounds of the invention show 2˜10-fold increases in activity compared to SAHA to suppress cytokine production. The compounds of the invention also caused markedly reduction in acute inflammation. Taken together, these results indicate that 1-arylsulfonyl-5-(N-hydroxyacrylamide)indolines and 1-arylsulfonyl-5-(N-hydroxyacrylamide)indoles HDAC inhibitors exhibit a potent anti-inflammatory activities. The also invention surprisingly found that the compounds of the invention are >10 times potent than the marketed HDAC inhibitor suberoylanilide hydroxamic acid (SAHA) on HDACs inhibition in rheumatoid arthritis. The compounds of the invention also have a longer half-life, higher systemic exposure and oral bioavailability than SAHA.

As used herein, except where the context requires otherwise, the term “comprise” and variations of the term, such as “comprising,” “comprises” and “comprised” are not intended to exclude other additives, components, integers or steps.

As used herein, except where the context requires otherwise, the method steps disclosed are not intended to be limiting nor are they intended to indicate that each step is essential to the method or that each step must occur in the order disclosed.

As used herein, the term “subject” is defined herein to include animals such as mammals, including, but not limited to, primates (e.g., humans), cows, sheep, goats, horses, dogs, cats, rabbits, rats, mice and the like. In specific embodiments, the subject is a human. The terms “subject” and “patient” are used interchangeably herein in reference, for example, to a mammalian subject, such as a human.

As used herein, the terms “treat,” “treating” and “treatment” refer to the eradication or amelioration of a disease or disorder, or of one or more symptoms associated with the disease or disorder. In certain embodiments, the terms refer to minimizing the spread or worsening of the disease or disorder resulting from the administration of one or more prophylactic or therapeutic agents to a subject with such a disease or disorder. In some embodiments, the terms refer to the administration of a compound or dosage form provided herein, with or without one or more additional active agent(s), after the diagnosis or onset of symptoms of the particular disease.

As used herein, the terms “prevent,” “preventing” and “prevention” refer to the prevention of the onset, recurrence or spread of a disease or disorder, or of one or more symptoms thereof. In certain embodiments, the terms refer to the treatment with or administration of a compound or an antibody or dosage form provided herein, with or without one or more other additional active agent(s), prior to the onset of symptoms, particularly to patients at risk of disease or disorders provided herein. The terms encompass the inhibition or reduction of a symptom of the particular disease. In this regard, the term “prevention” may be interchangeably used with the term “prophylactic treatment.

As used herein, the terms “co-administration” and “in combination with” include the administration of two or more therapeutic agents simultaneously, concurrently or sequentially within no specific time limits unless otherwise indicated. In one embodiment, the therapeutic agents are in the same composition or unit dosage form. In other embodiments, the therapeutic agents are in separate compositions or unit dosage forms.

As used herein, the term “effective amount” is the quantity of compound in which a beneficial outcome is achieved when the compound is administered to a subject or alternatively, the quantity of compound that possess a desired activity in-vivo or in-vitro. In the case of inflammatory disorders and immune disorders, a beneficial clinical outcome includes reduction in the extent or severity of the symptoms associated with the disease or disorder and/or an increase in the longevity and/or quality of life of the subject compared with the absence of the treatment. The precise amount of compound administered to a subject will depend on the type and severity of the disease or condition and on the characteristics of the subject, such as general health, age, sex, body weight and tolerance to drugs. It will also depend on the degree, severity and type of inflammatory disorder. The skilled artisan will be able to determine appropriate dosages depending on these and other factors.

As used herein, the term “aryl” means a monocyclic or polycyclic-aromatic ring or ring radical comprising carbon and hydrogen atoms. Examples of suitable aryl groups include, but are not limited to, phenyl, tolyl, anthacenyl, fluorenyl, indenyl, azulenyl, and naphthyl, as well as benzo-fused carbocyclic moieties such as 5,6,7,8-tetrahydronaphthyl. An aryl group can be unsubstituted or substituted with one or more substituents (including without limitation alkyl (preferably, lower alkyl or alkyl substituted with one or more halo), hydroxy, alkoxy (preferably, lower alkoxy), alkylthio, cyano, halo, amino, and nitro. In certain embodiments, the aryl group is a monocyclic ring, wherein the ring comprises 6 carbon atoms.

As used herein, the term “alkyl” means a saturated straight chain or branched non-cyclic hydrocarbon typically having from 1 to 6 carbon atoms. Representative saturated straight chain alkyls include methyl, ethyl, n-propyl, n-butyl, n-pentyl and n-hexyl; while saturated branched alkyls include isopropyl, sec-butyl, isobutyl, tert-butyl, isopentyl, 2-methylbutyl, 3-methylbutyl, 2-methylpentyl, 3-methylpentyl, 4-methylpentyl, 2,3-dimethylbutyl etc, and the like. Alkyl groups included in compounds of this invention may be optionally substituted with one or more substituents, such as amino, alkylamino, alkoxy, alkylthio, oxo, halo, acyl, nitro, hydroxyl, cyano, aryl, alkylaryl, aryloxy, arylthio, arylamino, carbocyclyl, carbocyclyloxy, carbocyclylthio, carbocyclylamino, heterocyclyl, heterocyclyloxy, heterocyclylamino, heterocyclylthio, and the like. In addition, any carbon in the alkyl segment may be substituted with oxygen, sulfur, or nitrogen.

The term “alkoxy,” as used herein, refers to an alkyl group which is linked to another moiety though an oxygen atom. Alkoxy groups can be substituted or unsubstituted.

As used herein, the term “alkenyl” means a straight chain or branched, hydrocarbon radical typically having from 2 to 10 carbon atoms and having at least one carbon-carbon double bond. Representative straight chain and branched alkenyls include vinyl, allyl, 1-butenyl, 2-butenyl, isobutylenyl, 1-pentenyl, 2-pentenyl, 3-methyl-1-butenyl, 1-methyl-2-butenyl, 2,3-dimethyl-2-butenyl, 1-hexenyl, 2-hexenyl, 3-hexenyl, 1-heptenyl, 2-heptenyl, 3-heptenyl, 1-octenyl, 2-octenyl, 3-octenyl, 1-nonenyl, 2-nonenyl, 3-nonenyl, 1-decenyl, 2-decenyl, 3-decenyl and the like. Alkenyl groups can be substituted or unsubstituted.

As used herein, the term “alkynyl” means a straight chain or branched, hydrocarbon radical typically having from 2 to 10 carbon atoms and having at lease one carbon-carbon triple bond. Representative straight chain and branched alkynyls include acetylenyl, propynyl, 1-butynyl, 2-butynyl, 1-pentynyl, 2-pentynyl, 3-methyl-1-butynyl, 4-pentynyl, -1-hexynyl, 2-hexynyl, 5-hexynyl, 1-heptynyl, 2-heptynyl, 6-heptynyl, 1-octynyl, 2-octynyl, 7-octynyl, 1-nonynyl, 2-nonynyl, 8-nonynyl, 1-decynyl, 2-decynyl, 9-decynyl and the like. Alkynyl groups can be substituted or unsubstituted.

As used herein, the term “cycloalkyl” means a saturated, mono- or polycyclic alkyl radical typically having from 3 to 10 carbon atoms. Representative cycloalkyls include cyclopropyl, cyclobutyl, cyclopentyl, cyclohexyl, cycloheptyl, cyclooctyl, cyclononyl, cyclodecyl, adamantly, decahydronaphthyl, octahydropentalene, bicycle[1.1.1]pentanyl, and the like. Cycloalkyl groups can be substituted or unsubstituted.

As used herein, the term “cycloalkenyl” means a cyclic non-aromatic alkenyl radical having at least one carbon-carbon double bond in the cyclic system and typically having from 5 to 10 carbon atoms. Representative cycloalkenyls include cyclopentenyl, cyclopentadienyl, cyclohexenyl, cyclohexadienyl, cycloheptenyl, cycloheptadienyl, cycloheptatrienyl, cyclooctenyl, cyclooctadienyl, cyclooctatrienyl, cyclooctatetraenyl, cyclononenyl, cyclononadienyl, cyclodecenyl, cyclodecadienyl and the like. Cycloalkenyl groups can be substituted or unsubstituted.

As used herein, the term “heterocycle” or “heterocyclyl” means a monocyclic or polycyclic heterocyclic ring (typically having 3- to 14-members) which is either a saturated ring or a unsaturated non-aromatic ring. A 3-membered heterocycle can contain up to 3 heteroatoms, and a 4- to 14-membered heterocycle can contain from 1 to about 8 heteroatoms. Each heteroatom is independently selected from nitrogen, which can be quaternized; oxygen; and sulfur, including sulfoxide and sulfone. The heterocycle may be attached via any heteroatom or carbon atom. Representative heterocycles include morpholinyl, thiomorpholinyl, pyrrolidinonyl, pyrrolidinyl, piperidinyl, piperazinyl, hydantoinyl, valerolactamyl, oxiranyl, oxetanyl, tetrahydrofuranyl, tetrahydropyranyl, tetrahydropyrindinyl, tetrahydropyrimidinyl, tetrahydrothiophenyl, tetrahydrothiopyranyl, and the like. A heteroatom may be substituted with a protecting group known to those of ordinary skill in the art, for example, the hydrogen on a nitrogen may be substituted with a tert-butoxycarbonyl group. Furthermore, the heterocyclyl may be optionally substituted with one or more substituents (including without limitation a halogen atom, an alkyl radical, or aryl radical). Only stable isomers of such substituted heterocyclic groups are contemplated in this definition. Heterocyclyl groups can be substituted or unsubstituted.

As used herein, the term “heteroaryl” means a monocyclic or polycyclic heteroaromatic ring (or radical thereof) comprising carbon atom ring members and one or more heteroatom ring members (such as, for example, oxygen, sulfur or nitrogen). Typically, the heteroaryl has from 5 to about 14 ring members in which at least 1 ring member is a heteroatom selected from oxygen, sulfur and nitrogen. In another embodiment, the heteroaryl is a 5 or 6 membered ring and may contain from 1 to about 4 heteroatoms. In another embodiment, the heteroaryl has a 7 to 14 ring members and may contain from 1 to about 7 heteroatoms. Representative heteroaryls include pyridyl, furyl, thienyl, pyrrolyl, oxazolyl, imidazolyl, indolizinyl, thiazolyl, isoxazolyl, pyrazolyl, isothiazolyl, pyridazinyl, pyrimidinyl, pyrazinyl, triazinyl, triazolyl, pyridinyl, thiadiazolyl, pyrazinyl, quinolyl, isoquinolyl, indazolyl, benzoxazolyl, benzofuryl, benzothiazolyl, indolizinyl, imidazopyridinyl, isothiazolyl, tetrazolyl, benzimidazolyl, benzoxazolyl, benzothiazolyl, benzothiadiazolyl, benzoxadiazolyl, indolyl, tetrahydroindolyl, azaindolyl, imidazopyridyl, quinozalinyl, purinyl, pyrrolo[2,3]pyrimidyl, pyrazolo[3,4]pyrimidyl or benzo(b)thienyl and the like. These heteroaryl groups may be optionally substituted with one or more substituents.

In one aspect, the invention provides a method for inhibiting cytokine release from a cell or a subject, comprising administering an effective amount of the compound having formula (I) or a pharmaceutically acceptable salt, prodrug or solvate thereof to the cell or the subject:

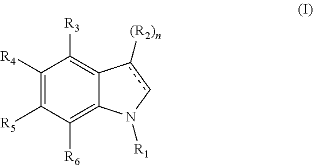

wherein [symbol] is a single bond or a double bond;

R₁ is SO₂Rₐ, wherein Rₐ is aryl unsubstituted or substituted by 1 to 3 substituent selected from the group consisting of: —OC₁₋₁₀alkyl, halogen, —NO₂, —NH₂, —OH, —C₁₋₆alkyl, —C₂₋₁₀alkenyl, —C₂₋₁₀alkynyl, —C₃₋₁₀cycloalkyl, —C₅₋₁₀cycloalkenyl, 6 to 10 membered aryl or 6 to 10 membered heteroaryl;

R₂, R₅ and R₆ are each independently H, —OC₁₋₁₀alkyl, halogen, —NO₂, —NH₂, —OH, —C₁₋₁₀alkyl, —C₂₋₁₀alkenyl or —C₂₋₁₀alkynyl; and

R₄ is H, C₁₋₁₀alkyl, C₂₋₁₀alkenyl, C₂₋₁₀alkynyl, aryl, 5 to 14 membered heteroaryl, C₃₋₁₀cycloalkyl, C₅₋₁₀cycloalkenyl, C₃₋₁₄heterocycloC₁₋₁₀alkyl, C₅₋₁₄heterocyclo C₂₋₁₀alkenyl, halo, cyano, nitro, OR_{b}, SR_{b}, S(O)R_{b}, CH═CH—C(O)NR_{c}R_{d}, NHC(O)—CH═CH—C(O)R_{b}, NHC(O)—CH═CH—C(O)NRcRd, SO₂NRcRd, OC(O)R_{b}, C(O)NR_{c}R_{d}, NRcRd, NHC(O)R_{b}, NHC(O)NR_{c}R_{d}, or NHC(S)Rc, in which each of R_{b}, R_{c}, and R_{d}, independently, is H, hydroxy, C₁₋₁₀alkoxy, C₆₋₁₀aryloxy, C₅₋₁₄heteroaryloxy, C₁₋₁₀alkyl, C₂₋₁₀alkenyl, C₂₋₁₀alkynyl, C₆₋₁₀aryl, C₅₋₁₄heteroaryl, C₃₋₁₀cycloalkyl, C₅₋₁₀cycloalkenyl, C₃₋₁₄heterocycloC₁₋₆alkyl, or C₅₋₁₄heterocycloC₂₋₁₀alkenyl.

In one embodiment, aryl is 6 to 10 membered aryl; C₁₋₁₀alkyl is C₁₋₄alkyl or C₁₋₆alkyl; C₂₋₁₀alkenyl is C₂₋₆alkenyl; or C₂₋₁₀alkynyl is C₂₋₆alkynyl.

In one embodiment, Rₐ is 6 to 10 membered aryl unsubstituted or substituted by 1 to 3 substituent selected from the group consisting of: —OC₁₋₆alkyl, halogen, —NO₂, —NH₂, or —OH. Preferably, Rₐ is phenyl. Preferably, Ra is phenyl substituted by one to three, same or different, —OCH3, halogen, NO₂ or NH₂.

In one embodiment, R₄ is CH═CH—C(O)NR_{c}R_{d}, NHC(O)—CH═CH—C(O)R_{b}, NHC(O)—CH═CH—C(O)NRcRd, NHC(O)R_{b}, NHC(O)NR_{c}R_{d}, or NHC(S)Rc.

In one embodiment, R₂, R₅ and R₆ are each independently H, halogen, —NO₂, —NH₂, or —OH.

In one embodiment, R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or —OH. Preferably, R₄ is CH═CH—C(O)NR_{c}R_{d}, and Rₐ is phenyl or naphthyl.

In one embodiment, when [symbol] is a double bond, R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or —OH. Preferably, R₄ is CH═CH—C(O)NR_{c}R_{d}, and Rₐ is phenyl or naphthyl.

In one embodiment, when [symbol] is a single bond, R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or OH. Preferably, Rₐ is phenyl or naphthyl unsubstituted or substituted by 1 to 3 substituent selected from the group consisting of: —OC₁₋₆alkyl, halogen, —NO₂, —NH₂, or OH. More preferably, Rₐ is phenyl or naphthyl unsubstituted or substituted by 1 to 2 substituent selected from the group consisting of: —OCH₃, halogen, —NO₂, —NH₂, or OH.

In one embodiment, the compound is one of the following compounds:

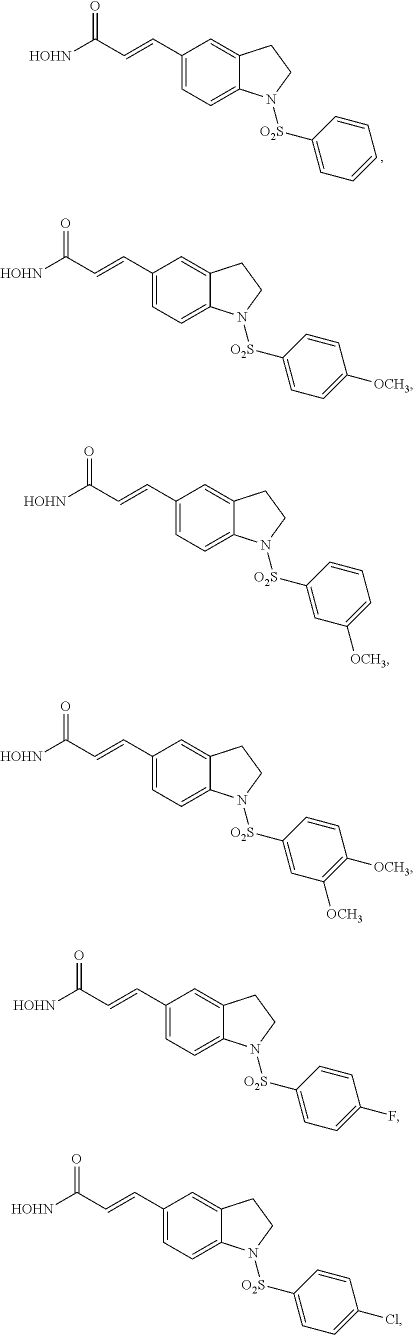

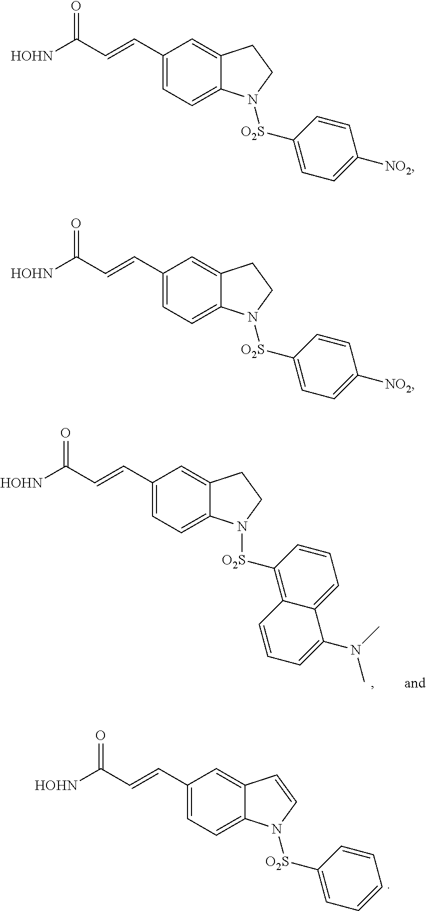

In another embodiment, the invention provides a method for inhibiting HDACs 1, 2, 3, and 8 in a cell or a subject, comprising administering an effective amount of the compound having formula (I) or a pharmaceutically acceptable salt, prodrug or solvate thereof to the cell or subject.

The “pharmaceutically acceptable salt” is a salt formed from an acid and a basic group of one of the compounds of any one of formulas (I) mentioned herein. Illustrative salts include, but are not limited, to sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate, and pamoate (i.e., 1,1′-methylene-bis-(2-hydroxy-3-naphthoate)) salts. The “pharmaceutically acceptable salt” also refers to a salt prepared from a compound of any one of formulas (I) having an acidic functional group and a pharmaceutically acceptable inorganic or organic base. Suitable bases include, but are not limited to, hydroxides of alkali metals such as sodium, potassium, and lithium; hydroxides of alkaline earth metal such as calcium and magnesium; hydroxides of other metals, such as aluminum and zinc; ammonia, and organic amines, such as unsubstituted or hydroxy-substituted mono-, di-, or trialkylamines; dicyclohexylamine; tributyl amine; pyridine; N-methyl, N-ethylamine; diethylamine; triethylamine; mono-, bis-, or tris-(2-hydroxy-lower alkyl amines), such as mono-, bis-, or tris-(2-hydroxyethyl)-amine, 2-hydroxy-tert-butylamine, or tris-(hydroxymethyl)methylamine, N,N,-di-lower alkyl-N-(hydroxy lower alkyl)-amines, such as N,N-dimethyl-N-(2-hydroxyethyl)amine, or tri-(2-hydroxyethyl)amine; N-methyl-D-glucamine; and amino acids such as arginine, lysine, and the like. The pharmaceutically acceptable salt also refers to a salt prepared from a compound of any one of formulas (I) having a basic functional group, such as an amino functional group, and a pharmaceutically acceptable inorganic or organic acid. Suitable acids include, but are not limited to, hydrogen sulfate, citric acid, acetic acid, oxalic acid, hydrochloric acid, hydrogen bromide, hydrogen iodide, nitric acid, phosphoric acid, isonicotinic acid, lactic acid, salicylic acid, tartaric acid, ascorbic acid, succinic acid, maleic acid, besylic acid, fumaric acid, gluconic acid, glucaronic acid, saccharic acid, formic acid, benzoic acid, glutamic acid, methanesulfonic acid, ethanesulfonic acid, benzenesulfonic acid, and p-toluenesulfonic acid.

As used herein, the term “pharmaceutically acceptable solvate,” is a solvate formed from the association of one or more solvent molecules to one or more molecules of a compound of any one of formulas (I). The term solvate includes hydrates (e.g., hemi-hydrate, mono-hydrate, dihydrate, trihydrate, tetrahydrate, and the like).

The compounds of this invention may be prepared by methods generally disclosed in U.S. patent application Ser. No. 12/912,260.

In one embodiment, the inhibition of cytokine release is associated with an inflammatory disease, particularly, a chronic inflammation disease. The inflammatory disease include, but not limited to, arthritis (such as rheumatoid arthritis, juvenile rheumatoid arthritis, osteoarthritis and psoriatic arthritis), synovitis, vasculitis, conditions associated with inflammation of the bowel (such as Crohn's disease, ulcerative colitis, inflammatory bowel disease and irritable bowel syndrome), atherosclerosis, multiple sclerosis, Alzheimer's disease, vascular dementia, pulmonary inflammatory diseases (such as asthma, chronic obstructive pulmonary disease and acute respiratory distress syndrome), fibrotic diseases (including idiopathic pulmonary fibrosis, cardiac fibrosis and systemic sclerosis (scleroderma)), inflammatory diseases of the skin (such as contact dermatitis, atopic dermatitis and psoriasis), systemic inflammatory response syndrome, sepsis, inflammatory and/or an autoimmune disorder (for example, autoimmune conditions of the liver (such as autoimmune hepatitis, primary biliary cirrhosis, alcoholic liver disease, sclerosing cholangitis, and autoimmune cholangitis), and/or the complications thereof. Preferably, the arthritis is osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease) or Still's disease. Preferably, the fibrosis is pulmonary fibrosis, liver fibrosis or renal fibrosis.

In general, compounds of the invention will be administered in therapeutically effective amounts by any of the usual modes known in the art, either singly or in combination with at least one other compound of this invention and/or at least one other conventional therapeutic agent for the disease being treated. A therapeutically effective amount may vary widely depending on the disease, its severity, the age and relative health of the animal being treated, the potency of the compound(s), and other factors. As anti-inflammatory agents, therapeutically effective amounts of compounds of this invention may range from 25-250 mg/Kg body weight/day, such as from 25 mg/Kg/day; for example, 25 mg/Kg/day. A person of ordinary skill in the art will be conventionally able, and without undue experimentation, having regard to that skill and to this disclosure, to determine a therapeutically effective amount of a compound for the treatment of inflammatory diseases such as arthritis and fibrosis.

In general, the compounds disclosed herein will be administered as pharmaceutical compositions by one of the following routes: oral, topical, systemic (e.g. transdermal, intranasal, or by suppository), or parenteral (e.g. intramuscular, subcutaneous, or intravenous injection). Compositions may take the form of tablets, pills, capsules, semisolids, powders, sustained release formulations, solutions, suspensions, elixirs, aerosols, or any other appropriate compositions; and comprise at least one compound of this invention in combination with at least one pharmaceutically acceptable excipient. Suitable excipients are well known to persons of ordinary skill in the art, and they, and the methods of formulating the compositions, may be found in such standard references as Remington: The Science and Practice of Pharmacy, A. Gennaro, ed., 20th edition, Lippincott, Williams & Wilkins, Philadelphia, Pa. Suitable liquid carriers, especially for injectable solutions, include water, aqueous saline solution, aqueous dextrose solution, and glycols.

In particular, the compound is administered in any particular dosage form. For example, the compound can be administered, orally, for example, as tablets, troches, lozenges, aqueous or oily suspension, dispersible powders or granules, emulsions, hard or soft capsules, or syrups or elixirs. Compositions intended for oral use may be prepared according to any method known in the art for the manufacture of pharmaceutical compositions and such compositions may contain one or more agents selected from the group consisting of sweetening agents, flavoring agents, coloring agents and preserving agents in order to provide pharmaceutically elegant and palatable preparations.

Tablets contain the compound in admixture with non-toxic pharmaceutically acceptable excipients that are suitable for the manufacture of tablets. These excipients may be for example, inert diluents, such as calcium carbonate, sodium carbonate, lactose, calcium phosphate or sodium phosphate; granulating and disintegrating agents, for example, maize starch or alginic acid; binding agents, for example, maize starch, gelatin or acacia, and lubricating agents, for example, magnesium stearate or stearic acid or tale. The tablets may be uncoated or they may be coated by known techniques to delay disintegration and absorption in the gastrointestinal tract and thereby provide a sustained action over a longer period. Formulations for oral use may also be presented as hard gelatin capsules wherein the compound is mixed with an inert solid diluent, for example, calcium carbonate, calcium phosphate or kaolin, or as soft gelatin capsules wherein the active ingredient is mixed with water or an oil medium, for example, peanut oil, liquid paraffin or olive oil.

Aqueous suspensions or oily suspensions may be formulated with excipients suitable for the manufacture of aqueous or oily suspensions.

The compounds of the invention can also be administered by injection or infusion, either subcutaneously or intravenously, or intramuscularly, or intrasternally, or intranasally, or by infusion techniques in the form of sterile injectable or oleaginous solution or suspension. The compound may be in the form of a sterile injectable aqueous or oleaginous solution or suspensions. These solution or suspensions may be formulated according to the known art using suitable solvent or dispersing of wetting agents and suspending agents that have been described above. The sterile injectable preparation may also be a sterile injectable solution or suspension in a non-toxic parenterally-acceptable diluent or solvent for example, as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oils may be conventionally employed including synthetic mono- or diglycerides. In addition fatty acids such as oleic acid find use in the preparation of injectables. Dosage regimens can be adjusted to provide the optimum therapeutic response. For example, several divided dosages may be administered daily or the dosage may be proportionally reduced as indicated by the exigencies of the therapeutic situation.

It is especially advantageous to formulate the compounds in dosage unit form for ease of administration and uniformity of dosage. Dosage unit form as used herein refers to physically discrete units suited as unitary dosages for the subjects to be treated; each containing a therapeutically effective quantity of the compound and at least one pharmaceutical excipient. A drug product will comprise a dosage unit form within a container that is labelled or accompanied by a label indicating the intended method of treatment, such as the treatment of an inflammatory disease such as arthritis and fibrosis.

### EXAMPLE

### Chemical Synthesis

Scheme 1 describes the synthesis of 1-arylsulfonyl-5-(N-hydroxyacrylamide)indolines beginning with commercially available indole-carboxylate 16. Treatment of 16 with sodium cyanoborohydride in the presence of acetic acid yielded indoline 17. The resulting product was reacted with various benzenesulfonyl chlorides to provide 18a-h. The ester functionalities were reduced by LAH followed by oxidation with PDC, yielding the corresponding aldehydes, 19a-h. Subsequently, the resulting products were subjected to the Wittig reaction with methyl (triphenylphosphoranylidene)acetate followed by conversion into acrylic acids (20a-h) by treatment with lithium hydroxide. The corresponding acrylic acids were reacted with NH₂OTHP to afford protected hydroxamates followed by TFA deprotection, yielding compounds 7-15.

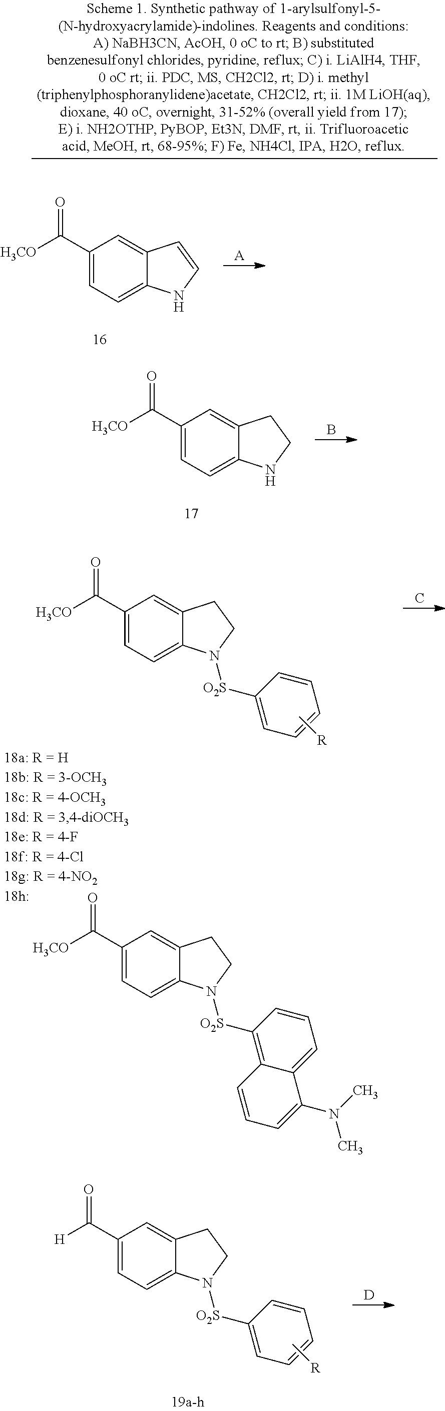

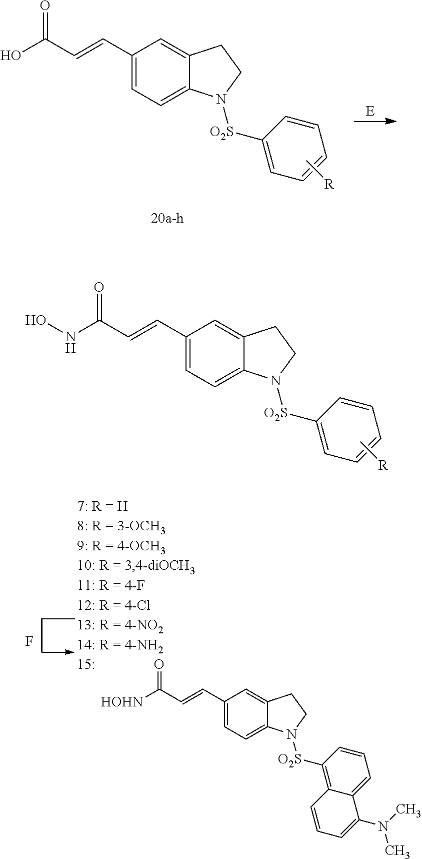

General procedures for preparation of 1-arylsulfonyl-5-(N-hydroxyacrylamide)indolines (7-15).

### Example 1

### 3-[1-(4-methoxybenzenesulfonyl)-2,3-dihydro-1H-indol-5-yl]-N-Hydroxyacrylamide 9 (hereafter referred to as “MPT0G009”)

To a solution of 16 (0.30 g, 1.71 mmol) in AcOH (2 mL) was added sodium cyanoborohydride (0.16 g, 2.57 mmol) at 0° C., and allowed to stir at room temperature for 2 h. The reaction was quenched with water at 0° C., concentrated NaOH was added up to pH 10. The aqueous layer was extracted with CH₂Cl₂ (15 mL×3). The combined organic layer was dried over anhydrous MgSO₄ and purified by chromatography over silica gel to afford 17 as a yellow solid (92% yield; 1:2 EtOAc/n-hexane): ¹H NMR (500 MHz, CDCl₃) δ 3.06 (t, J=8.5 Hz, 2H), 3.65 (t, J=8.5 Hz, 2H), 3.84 (s, 3H), 6.54 (dd, J=8.6, 4.7 Hz, 1H), 7.75-7.76 (m, 2H).

To a solution of 17 (0.28 g, 1.58 mmol) in pyridine (2 mL) was added 4-methoxybenzenesulfonyl chloride (0.32 g, 1.58 mmol) and heated to reflux for 6 h. The reaction mixture was purified by chromatography over silica gel to afford 18c as a white solid (85% yield; 1:1 EtOAc/n-hexane): ¹H NMR (300 MHz, CDCl₃) δ 2.96 (t, J=8.7 Hz, 2H), 3.80 (s, 3H), 3.85 (s, 3H), 3.93 (t, J=8.7 Hz, 2H), 6.89 (d, J=8.7 Hz, 2H), 7.62 (d, J=8.4 Hz, 1H), 7.20-7.76 (m, 3H), 7.88 (d, J=8.4 Hz, 1H).

To a solution of 18c (0.45 g, 1.26 mmol) in THF (10 mL), LAH (0.10 g, 2.52 mmol) was added at 0° C. The reaction was warmed to room temperature and stirred for 2 h. The reaction was quenched with water followed by extraction with CH₂Cl₂ (15 mL×3). The combined organic layer was dried over anhydrous MgSO₄ and purified by silica gel chromatography (1:1; EtOAc/n-hexane) to afford a brown solid. A solution of the resulting solid in CH₂Cl₂ (10 mL), PDC (0.63 g, 1.66 mmol) and MS (0.63 g) was stirred at room temperature for 1 h. The reaction was filtered through celite and the filtrate was purified by chromatography over silica gel to afford 19c (62% yield; 1:1 EtOAc/n-hexane): ¹H NMR (300 MHz, CDCl₃) δ 3.04 (t, J=8.4 Hz, 2H), 3.83 (s, 3H), 3.98 (t, J=8.4 Hz, 2H), 6.93 (d, J=9.0 Hz, 2H), 7.61 (s, 1H), 7.70-7.72 (m, 2H), 7.78 (d, J=9.0 Hz, 2H), 9.84 (s, 1H).

To a solution of 19c (0.20 g, 0.66 mmol) in CH₂Cl₂ (15 mL), methyl (triphenylphosphoranylidene)acetate (0.27 g, 0.80 mmol) was added and allowed to stir at room temperature for 6 h. The reaction mixture was quenched with water and extracted with CH₂Cl₂ (25 mL×3). The combined organic layer was dried over anhydrous MgSO₄ and concentrated under reduced pressure to give a yellow residue. To a solution of the crude adduct in dioxane (15 mL), 1M LiOH_{(aq)} (3.4 mL) was added and stirred at 40° C. for 6 h. The reaction was acidified by concentrated HCl to give the precipitate which was recrystallized in MeOH to afford 20c as a white solid (87%; overall 46% yield from 17): ¹H NMR (500 MHz, CD₃OD) δ 2.91 (t, J=8.5 Hz, 2H), 3.92 (t, J=8.5 Hz, 2H), 6.33 (d, J=15.9 Hz, 1H), 7.00 (d, J=8.9 Hz, 2H), 7.38 (s, 1H), 7.40 (d, J=8.4 Hz, 1H), 7.55-7.58 (m, 2H), 7.74-7.76 (m, 2H).

To a solution of 21c (0.2 g, 0.56 mmol), PyBOP (0.31 g, 0.59 mmol), triethylamine (0.19 ml, 1.34 mmol) in DMF (2 mL), NH₂OTHP (0.08 g, 0.67 mmol) was added and stirred at room temperature. After being stirred for 2 h, the reaction was quenched with water, followed by extraction with EtOAc (15 mL×3). The combined organic layer was dried over anhydrous MgSO₄ and concentrated under reduced pressure. The residue was purified by silica gel chromatography (30:1:1%; CH₂Cl₂/CH₃OH/NH_{3(aq)} to give a white solid, which was treated with TFA (1.8 mL, 24.2 mmol) in the presence of CH₃OH (33 mL) and stirred overnight at room temperature. The reaction mixture was concentrated under reduced pressure to give a white residue, which was recrystallized by CH₃OH to afford 9 as a white solid (96% yield): mp: 158-160° C.; ¹H NMR (500 MHz, CD₃OD) δ 2.91 (t, J=8.5 Hz, 2H), 3.81 (s, 3H), 3.92 (t, J=8.5 Hz, 2H), 6.32 (d, J=15.5 Hz, 1H), 7.00 (d, J=9.0 Hz, 2H), 7.32 (s, 1H), 7.37 (d, J=8.0 Hz, 1H), 7.47 (d, J=15.5 Hz, 1H), 7.56 (d, J=8.0 Hz, 1H), 7.74 (d, J=9.0 Hz, 2H); MS (EI) m/z: 373 (M⁺, 3.33%), 98 (100%); HRMS (EI) for C₁₈H₁₈N₂O₅S (M⁺) calcd 374.0936. Found 374.0939.

### Example 2

### 3-(1-Benzenesulfonyl-2,3-dihydro-1H-indol-5-yl)-N-hydroxyacrylamide 7

The title compound was obtained in 97% overall yield from compound 20a in a manner similar to that described for the preparation of 9: mp 128-130° C.; ¹H NMR (500 MHz, CD₃OD) δ 2.91 (t, J=8.5 Hz, 2H), 3.95 (t, J=8.5 Hz, 2H), 6.32 (d, J=15.5 Hz, 1H), 7.32 (s, 1H), 7.38 (d, J=8.5 Hz, 1H), 7.46 (d, J=15.5 Hz, 1H), 7.51 (dd, J=7.5, 8.0 Hz, 2H), 7.58 (d, J=8.5 Hz, 1H), 7.61 (dd, J=1.0, 8.0 Hz, 1H), 7.81 (d, J=7.5 Hz, 2H); MS (EI) m/z: 344 (M⁺, 3.21%), 170 (100%); HRMS (EI) for C₁₇H₁₆N₂O₄S (M⁺) calcd 344.0831. Found 344.0829.

### Example 3

### 3-[1-(3-Methoxybenzenesulfonyl)-2,3-dihydro-1H-indol-5-yl]-N-hydroxyacrylamide 8

The title compound was obtained in 95% overall yield from compound 20b in a manner similar to that described for the preparation of 9: mp: 156-157° C.; ¹H NMR (300 MHz, CD₃OD) δ 2.82 (t, J=8.5 Hz, 2H), 3.65 (s, 3H), 3.82 (t, J=8.5 Hz, 2H), 6.18 (d, J=15.5 Hz, 1H), 6.97-7.00 (m, 1H), 7.14-7.15 (m, 2H), 7.23-7.28 (m, 3H), 7.42 (d, J=15.5 Hz, 1H), 7.49 (d, J=8.5 Hz, 1H); MS (EI) m/z: 413 (M⁺+K). Anal. Calcd for C₁₈H₁₈N₂O₅S.1.5 H₂O: C, 53.86; H, 5.27; N, 6.98. Found: C, 53.73; H, 5.12; N, 6.70.

### Example 4

### 3-[1-(3,4-Dimethoxybenzenesulfonyl)-2,3-dihydro-1H-indol-5-yl]-N-hydroxyacrylamide 10

The title compound was obtained in 68% overall yield from compound 20d in a manner similar to that described for the preparation of 9: mp: 192-193° C.; ¹H NMR (500 MHz, CD₃OD) δ 2.90 (t, J=8.5 Hz, 2H), 3.72 (s, 3H), 3.85 (s, 3H), 3.93 (t, J=8.5 Hz, 2H), 6.35 (d, J=15.5 Hz, 1H), 7.06 (d, J=8.5 Hz, 1H), 7.19 (d, J=1.5 Hz, 1H), 7.36 (s, 1H), 7.42 (d, J=8.5 Hz, 1H), 7.45 (d, J=8.5 Hz, 1H), 7.48 (d, J=15.5 Hz, 1H), 7.61 (d, J=8.5 Hz, 1H); MS (EI) m/z: 389 (M⁺−15, 55%), 170 (100%); HRMS (EI) for C₁₉H₂₀N₂O₆S (M⁺) calcd 402.1042. Found 404.1042.

### Example 5

### 3-[1-(4-Fluorobenzenesulfonyl)-2,3-dihydro-1H-indol-5-yl]-N-hydroxyacrylamide 11

The title compound was obtained in 95% overall yield from compound 20e in a manner similar to that described for the preparation of 9: mp: 129-131° C.; ¹H NMR (500 MHz, CD₃OD) δ 2.93 (t, J=8.5 Hz, 2H), 3.95 (t, J=8.5 Hz, 2H), 6.80 (d, J=15.5 Hz, 1H), 7.25 (dd, J=8.5, 9.0 Hz, 2H), 7.33 (s, 1H), 7.38 (d, J=8.5 Hz, 1H), 7.40 (d, J=15.5 Hz, 1H), 7.56 (d, J=8.5 Hz, 1H), 7.87 (dd, J=5.5, 8.5 Hz, 2H); MS (EI) m/z: 362 (M⁺, 25%), 132 (100%); HRMS (EI) for C₁₇H₁₅FN₂O₄S (M⁺) calcd 362.0737. Found 362.0739.

### Example 6

### 3-[1-(4-Chlorobenzenesulfonyl)-2,3-dihydro-1H-indol-5-yl]-N-hydroxyacrylamide 12

The title compound was obtained in 94% overall yield from compound 20f in a manner similar to that described for the preparation of 9: mp: 159-160° C.; ¹H NMR (300 MHz, CD₃OD) δ 2.93 (t, J=8.5 Hz, 2H), 3.96 (t, J=8.5 Hz, 2H), 6.33 (d, J=15.5 Hz, 1H), 7.34 (s, 1H), 7.39 (d, J=8.5 Hz, 1H), 7.47 (d, J=15.5 Hz, 1H), 7.53 (d, J=8.5 Hz, 2H), 7.57 (d, J=8.0 Hz, 1H), 7.80 (dd, J=8.5 Hz, 1H); MS (EI) m/z: 378 (M⁺); Anal. Calcd for C₁₇H₁₅ClN₂O₄S.0.5 H₂O: C, 52.65; H, 4.16; N, 7.22. Found: C, 52.45; H, 4.26; N, 7.01.

### Example 7

### 3-[1-(4-Nitrobenzenesulfonyl)-2,3-dihydro-1H-indol-5-yl]-N-Hydroxyacrylamide 13

The title compound was obtained in 85% overall yield from compound 20g in a manner similar to that described for the preparation of 9: mp 163-165° C.; ¹H NMR (500 MHz, CD₃OD) δ 2.95 (t, J=8.5 Hz, 2H), 4.02 (t, J=8.5 Hz, 2H), 6.33 (d, J=16.0 Hz, 1H), 7.34 (s, 1H), 7.40 (d, J=8.0 Hz, 1H), 7.46 (d, J=16.0 Hz, 1H), 7.59 (d, J=8.0 Hz, 1H), 8.07 (d, J=9.0 Hz, 2H), 8.34 (d, J=9.0 Hz, 2H); MS (EI) m/z: 389 (M⁺); HRMS (EI) for C₁₇H₁₅N₃O₆S (M⁺) calcd, 389.0682. Found, 389.0680. Anal. Calcd for C₁₇H₁₅N₃O₆S.0.5 H₂O: C, 51.25; H, 4.05; N, 10.55. Found: C, 51.28; H, 4.27; N, 10.73.

### Example 8

### 3-[1-(4-Aminobenzenesulfonyl)-2,3-dihydro-1H-indol-5-yl]-N-hydroxyacrylamide 14

A mixture of 13 (0.3 g, 0.77 mmol), iron powder (0.13 g, 2.31 mmol) and ammonium chloride (0.08 g, 1.54 mmol) in isopropyl alcohol (8 mL) and water (1.5 mL) was heated to reflux for 4 h. The reaction was quenched with water and extracted with CH₂Cl₂ (25 mL×3). The combined organic layer was dried over anhydrous MgSO₄ and concentrated under reduced pressure. The reaction mixture was purified by chromatography over silica gel to afford 14 (47% yield; 10:1:1%; CH₂Cl₂/CH₃OH/NH_{3(aq)}): mp: 97-99° C.; ¹H NMR (500 MHz, CD₃OD) δ 2.90 (t, J=8.5 Hz, 2H), 3.87 (t, J=8.5 Hz, 2H), 6.49 (d, J=16.0 Hz, 1H), 6.58 (dd, J=2.0, 9.0 Hz, 2H), 7.34 (s, 1H), 7.37 (d, J=8.5 Hz, 1H), 7.45 (d, J=16.0 Hz, 1H), 7.45 (d, J=9.0 Hz, 2H), 7.53 (d, J=8.5 Hz, 1H); MS (EI) m/z: 341 (M⁺−18, 71%), 156 (100%); HRMS (EI) for C₁₇H₁₇N₃O₄S (M⁺) calcd 359.0940. Found 359.0940.

### Example 9

### 3-[1-(5-Dimethylaminonaphthalene-1-sulfonyl)-2,3-dihydro-1H-indol-5-yl]-N-hydroxyacrylamide 15

The title compound was obtained in 47% overall yield from compound 20h in a manner similar to that described for the preparation of 9: mp: 142-143° C.; ¹H NMR (300 MHz, CD₃OD) δ 2.85 (s, 6H), 2.92 (t, J=8.7 Hz, 2H), 4.04 (t, J=8.7 Hz, 2H), 6.32 (d, J=15.9 Hz, 1H), 7.23 (d, J=7.5 Hz, 1H), 7.35 (d, J=8.4 Hz, 1H), 7.44-7.60 (m, 4H), 8.23 (dd, J=1.5, 7.5 Hz, 1H), 8.35 (d, J=8.7 Hz, 1H), 8.58 (d, J=8.7 Hz, 1H). MS (EI) m/z: 435 (M⁺−2). Anal. Calcd for C₂₃H₂₃N₃O₄S.C₂H₅OH: C, 62.09; H, 6.04; N, 8.69. Found: C, 62.14; H, 5.96; N, 8.22.

### Example 10

### 3-(1-Benzenesulfonyl-2,3-dihydro-1H-indol-5-yl)-acrylic acid 20a

The title compound was obtained in 52% overall yield from compound 17 in a manner similar to that described for the preparation of 20c: ¹H NMR (500 MHz, CD₃OD) δ 2.92 (t, J=8.5 Hz, 2H), 3.96 (t, J=8.5 Hz, 2H), 6.33 (d, J=16.0 Hz, 1H), 7.38 (s, 1H), 7.41 (d, J=8.5 Hz, 1H), 7.52 (d, J=7.7 Hz, 1H), 7.55 (d, J=16.0 Hz, 1H), 7.59-7.64 (m, 3H), 7.82 (d, J=7.7 Hz, 2H).

### Example 11

### 3-[1-(3-Methoxybenzenesulfonyl)-2,3-dihydro-1H-indol-5-yl]-acrylic acid 20b

The title compound was obtained in 45% overall yield from compound 17 in a manner similar to that described for the preparation of 20c: ¹H NMR (300 MHz, CD₃Cl₃) δ 2.96 (t, J=8.4 Hz, 2H), 3.77 (s, 3H), 3.96 (t, J=8.7 Hz, 2H), 6.29 (d, J=16.0 Hz, 1H), 7.08-7.13 (m, 1H), 7.28-7.41 (m, 5H), 7.60 (d, J=16.0 Hz, 1H), 7.65 (s, 1H).

### Example 12

### 3-[1-(3,4-Dimethoxybenzenesulfonyl)-2,3-dihydro-1H-indol-5-yl]-acrylic acid 20d

The title compound was obtained in 52% overall yield from compound 17 in a manner similar to that described for the preparation of 20c: ¹H NMR (500 MHz, CD₃OD) δ 2.89 (t, J=8.4 Hz, 2H), 3.69 (s, 3H), 3.84 (s, 3H), 3.93 (t, J=8.4 Hz, 2H), 6.33 (d, J=15.9 Hz, 1H), 7.03 (d, J=8.5 Hz, 1H), 7.18 (d, J=1.8 Hz, 1H), 7.39 (s, 1H), 7.43 (dd, J=8.5, 1.8 Hz, 2H), 7.56 (d, J=15.9 Hz, 1H), 7.61 (d, J=8.5 Hz, 1H).

### Example 13

### 3-[1-(4-Fluorobenzenesulfonyl)-2,3-dihydro-1H-indol-5-yl]-acrylic acid 20e

The title compound was obtained in 50% overall yield from compound 17 in a manner similar to that described for the preparation of 20c: ¹H NMR (500 MHz, CD₃OD) δ 2.94 (t, J=8.4 Hz, 2H), 3.96 (t, J=8.4 Hz, 2H), 6.33 (d, J=15.9 Hz, 1H), 7.24-7.27 (m, 1H), 7.40 (s, 1H), 7.41 (d, J=8.5 Hz, 1H), 7.55-7.60 (m, 2H), 7.87-7.90 (m, 2H).

### Example 14

### 3-[1-(4-Chlorobenzenesulfonyl)-2,3-dihydro-1H-indol-5-yl]-acrylic acid 20f

The title compound was obtained in 46% overall yield from compound 17 in a manner similar to that described for the preparation of 20c: ¹H NMR (300 MHz, CD₃OD) δ 2.91 (t, J=8.7 Hz, 2H), 3.95 (t, J=8.4 Hz, 2H), 6.39 (d, J=16.2 Hz, 1H), 7.27-7.37 (m, 3H), 7.50-7.57 (m, 3H), 7.77-7.82 (m, 2H).

### Example 15

### 3-[1-(4-Nitrobenzenesulfonyl)-2,3-dihydro-1H-indol-5-yl]-acrylic acid 20g

The title compound was obtained in 31% overall yield from compound 17 in a manner similar to that described for the preparation of 20c: ¹H NMR (500 MHz, CD₃OD) δ 2.97 (t, J=8.5 Hz, 2H), 3.99 (t, J=8.5 Hz, 2H), 6.27 (d, J=16.0 Hz, 1H), 7.32 (s, 1H), 7.37 (d, J=8.6 Hz, 1H), 7.53-7.60 (m, 2H), 8.01 (d, J=8.8 Hz, 2H), 8.31 (d, J=8.8 Hz, 2H).

### Example 16

### 3-[1-(5-Dimethylaminonaphthalene-1-sulfonyl)-2,3-dihydro-1H-indol-5-yl]-acrylic acid 20h

The title compound was obtained in 45% overall yield from compound 17 in a manner similar to that described for the preparation of 20c: ¹H NMR (500 MHz, CD₃OD) δ 2.84 (s, 6H), 2.92 (t, J=8.5 Hz, 2H), 4.04 (t, J=8.5 Hz, 2H), 6.35 (d, J=16.0 Hz, 1H), 7.22 (d, J=7.5 Hz, 1H), 7.38 (d, J=3.0 Hz, 2H), 7.44-7.48 (m, 2H), 7.54-7.58 (m, 2H), 8.23 (dd, J=7.5, 1.0 Hz, 1H), 8.30 (d, J=9.0 Hz, 1H), 8.57 (d, J=8.5 Hz, 1H).

### Biological Evaluations

### Materials

The non-conjugated primary antibodies used were specific for iNOS was purchased from Santa Cruz Biotechnology (Santa Cruz, Calif., USA); COX-2 and β-actin were purchased from Epitomics Inc. (Burlingame, Calif., USA). Horseradish peroxidase (HRP)-conjugated goat anti-mouse or anti-rabbit IgG antibodies were obtained from Jackson ImmunoResearch Inc. (Cambridgeshire, UK).

### Cell Culture

Mouse macrophage cell line RAW264.7 was obtained from the Bioresource Collection and Research Center. Cells were cultured in Dulbecco's modified Eagle's medium (DMEM; Gibco Laboratories Inc.) supplemented with 10% (v/v) fetal bovine serum (FBS; Invitrogen™ Life Technologies, Carlsbad, Calif., USA), 100 U/mL of penicillin, and 100 μg/mL of streptomycin (Biological Industries, Kibbutz Beit Haemek, Israel) at 37° C. in a humidified atmosphere of 5% CO₂ in air.

WI-38 cells, a normal human embryonic lung fibroblast cell line, were obtained from American Type Culture Collection (Manassas, Va.). Cells were grown in MEM nutrient mixture, containing 10% FCS, 2 mM L-glutamine, 0.1 mM NEAA, 1 mM sodium pyruvate, 50 U/ml penicillin G, and 100 μg/ml streptomycin, in a humidified 37° C. incubator with 5% CO₂ Cells were used between passages 18 and 30 for all experiments. After reaching confluence, cells were seeded onto 6-cm dishes for immunoblotting.

### HeLa Nuclear Extract HDAC Activity Assay

The HeLa nuclear extract HDAC activity was measured by using the HDAC Fluorescent Activity Assay Kit (BioVision, CA) according to manufacturer's instructions. Briefly, the HDAC fluorometric substrate and assay buffer were added to HeLa nuclear extracts in a 96-well format and incubated at 37° C. for 30 min. The reaction was stopped by adding lysine developer, and the mixture was incubated for another 30 min at 37° C. Additional negative controls included incubation without the nuclear extract, without the substrate, or without both. TSA at 1 μM served as the positive control. A fluorescence plate reader with excitation at 355 nm and emission at 460 nm was used to quantify HDAC activity.

### Nitric Oxide (NO) Assay

RAW 264.7 cells (1×10⁶) were plated and pretreated with the indicated concentrations of compound 9 for 1 h, and subjected to stimulation with LPS (25 ng/mL) for 24 h, and then 100 μL of Griess reagent (1% sulfanilamide in 5% phosphoric acid and 0.1% naphthylethylenediamine dihydrochloride) was mixed with 100 μL of the cell supernatant and the optical density at 550 nm was measured. Nitrite concentration was determined using a dilution of sodium nitrite as a standard.

### PGE₂ Determination

To investigate the effect of compound 9 and SAHA on PGE₂ levels in LPS stimulated cells, RAW 264.7 cells (1×10⁶) were treated in the presence or absence of test compounds for 1 h, and then stimulated with LPS (25 ng/mL) for 24 h at 37° C. The concentrations of PGE₂ in the supernatants of RAW 264.7 cell cultures were determined using an EIA kit (R&D Systems, Minneapolis, Minn., USA).

### IL-6 and TNF-α Determination

To determine the effect of compound 9 and SAHA on the production of cytokines IL-6 and TNF-α from LPS-stimulated cells, RAW 264.7 cells (1×10⁶) were plated and pretreated in the presence or absence of compound 9 and SAHA for 1 h, and then stimulated with LPS (25 ng/mL) for 24 h at 37° C. Supernatants were collected and the concentrations of cytokines IL-6 and TNF-α were measured by ELISA kit.

### Carrageenan-Induced Hind Paw Edema

Animal experiments were approved by the Institutional Animal Care and Use Committee of National Taiwan University College of Medicine (IACUC number: 20120226). Animals were divided into four groups (n=5). 0.5% (w/v) suspension of carrageenan in normal saline was administered to male Wistar rats (7-weeks) by intradermal injection into the base of the right hind paw. One hour prior to carrageenan injection, rats were oral administration of vehicle (1% carboxymethyl cellulose and 0.5% Tween 80) or a fine suspension of Compound 9 (25 mg/kg), SAHA (200 mg/kg) in vehicle. A positive control group was included in which rats were pretreated with 5 mg/kg Indomethacin. Three hours after carrageenan administration, the thickness and volume of the right hind paw were measured by digital caliper and digital plethysmometer (Diagnostic & Research Instruments CO., Ltd, Taipei, Taiwan), respectively.

### Western Blot Analysis

Western blot analyses were performed as described previously (Chen B C, Chang Y S, Kang J C, Hsu M J, Sheu J R, Chen T L, et al. Peptidoglycan induces nuclear factor-kappaB activation and cyclooxygenase-2 expression via Ras, Raf-1, and ERK in RAW 264.7 macrophages. J Biol Chem 2004; 279:20889-97). Briefly, WI-38 lung fibroblasts were cultured in 6-cm dishes. After reaching confluence, cells were pretreated with specific inhibitors (E028 and G009) as indicated for 30 min, and then treated with the vehicle (H₂O) or 10 ng/ml TGF-β for 2 h (CTGF assay) or 24 h (collagen I assay). Whole-cell lysates (30 rig) were subjected to 12% (CTGF) or 8% (collagen I) SDS-PAGE, and transferred onto a polyvinylidene difluoride membrane which was then incubated in TBST buffer (150 mM NaCl, 20 mM Tris-HCl, and 0.02% Tween 20; pH 7.4) containing 5% BSA. Proteins were visualized by specific primary antibodies and then incubated with HRP-conjugated secondary antibodies. The immunoreactivity was detected using enhanced chemiluminescence (ECL) following the manufacturer's instructions. Quantitative data were obtained using a computing densitometer with scientific imaging systems (Kodak, Rochester, N.Y.).

### Statistical Analyses

Results are expressed as the mean±SEM for the indicated number of separate experiments. Means were checked for statistical difference using 1-test and P-values<0.05 were considered significant.

### Example 17

### MPT0G009 Inhibits HDAC Isoform Activity

Using kits that contained different recombinant HDAC isoforms, we evaluated the ability of MPT0G009 to inhibit HDAC-mediated deacetylation of lysine residues on the substrates that were provided. As shown in Table 1, MPT0G009 demonstrated potent inhibitory activity for class I HDACs 1, 2, 3, and 8 and for class IIb HDAC6 but not for class Ha HDAC4, with IC₅₀ values of 4.62, 5.16, 1.91, 22.48, 8.43, and >10⁴ nM, respectively.

TABLE 1
IC₅₀ values for MPT0G009 for different recombinant HDAC isoforms

| | IC₅₀ (nM) of HDAC isoforms enzyme^{a} | | | | | |
|---|---|---|---|---|---|---|
| Compound | HDAC1 | HDAC2 | HDAC3 | DHAC8 | HDAC4 | HDAC6 |
| MPT0G009 | 4.62 ± 0.81 | 5.16 ± 0.76 | 1.91 ± 0.22 | 22.48 ± 2.16 | >10⁴ | 8.43 ± 0.72 |

^{a}Data represent the mean ± SEM from three replicate experiments.

### Example 18

### Inhibition of LPS-Induced Production of Nitric Oxide, IL-6, PGE₂, and TNF-α

The effect of the synthesized 5-(N-hydroxyacrylamide)-1-benzenesulfonylindolines 7-15, 5-(N-hydroxyacrylamide)-1-benzenesulfonylindole 6, and reference compound 1 on inflammatory factors was summarized in Table 2. Lipopolysacharide (LPS) stimulated RAW 264.7 macrophages were treated with test compounds at the indicated concentrations and the IC₅₀ value of the compounds for inhibiting inflammatory factors nitric oxide (NO), interleukin-6 (IL-6), prostaglandin E₂ (PGE₂), and tumor necrosis factor-α (TNF-α) releasing were measured by ELISA kit (Table 2). Compound 6 possessing an indole nucleus exhibited comparable activity to 1. With the exception of 14, the conversion of central skeleton from indole to indoline led to overall improvement of activity decreasing the induction of inflammatory factors. The 4′-amino substitution of 14 caused a dramatic loss of potency, which is correlated with the result of HeLa nuclear HDAC enzyme inhibition. The replacement of N,N-dimethylnaphthalene (15) led to slight decrease of activity; however, it is still better than 6 and 1. Among all indoline analogues, 9 having a 4′-OMe group exhibited the most potent inhibiting the inflammatory factors secretion. 9 reduced the expression of NO, IL-6, PGE₂, and TNF-α with IC₅₀ values of 1.07, 0.01, 0.52, and 0.52 μM, respectively. (Table 2)

### Compound 6

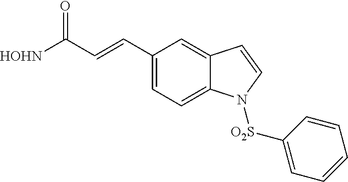

### Reference Compound 1 (Vorinostat Also Known as Suberanilohydroxamic Acid (SAHA))

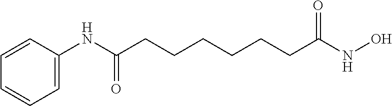 [SMILES: O=C(CCCCCCC(=O)Nc1ccccc1)NO]

TABLE 2
IC₅₀ values (μM ± SEM)^{[a]} of test compounds inhibiting
LPS-induced inflammatory factors expression
in RAW 264.7 macrophages.

| compd | NO | IL-6 | PGE₂ | TNF-α |
|---|---|---|---|---|
| 6²² | >10 | 0.17 ± 1.1E−3 | 5.13 ± 0.48 | 3.73 ± 0.26 |
| 7 | 4.64 ± 0.12 | 0.02 ± 4.0E−4 | 1.68 ± 0.27 | 0.99 ± 0.07 |
| 8 | 7.35 ± 0.83 | 0.02 ± 3.2E−4 | 1.36 ± 0.29 | 1.09 ± 0.12 |
| 9 | 1.07 ± 0.26 | 0.01 ± 1.2E−4 | 0.52 ± 0.06 | 0.52 ± 0.09 |
| 10 | 7.31 ± 0.66 | 0.01 ± 6.4E−4 | 1.90 ± 0.21 | 0.62 ± 0.12 |
| 11 | 4.41 ± 0.31 | 0.03 ± 8.7E−4 | 1.31 ± 0.16 | 1.04 ± 0.25 |
| 12 | 5.49 ± 0.48 | 0.02 ± 9.3E−4 | 1.04 ± 0.15 | 1.01 ± 0.11 |
| 13 | 3.11 ± 0.37 | 0.02 ± 3.2E−4 | 1.76 ± 0.26 | 1.37 ± 0.18 |
| 14 | >10 | >10 | >10 | >10 |
| 15 | >10 | 0.09 ± 9.3E−4 | 2.70 ± 0.43 | 2.11 ± 0.28 |
| SAHA,1 | >10 | 0.15 ± 1.0E−3 | 3.22 ± 0.51 | 1.19 ± 0.05 |

^{[a]}Data represent mean ± SEM from three independent experiments.

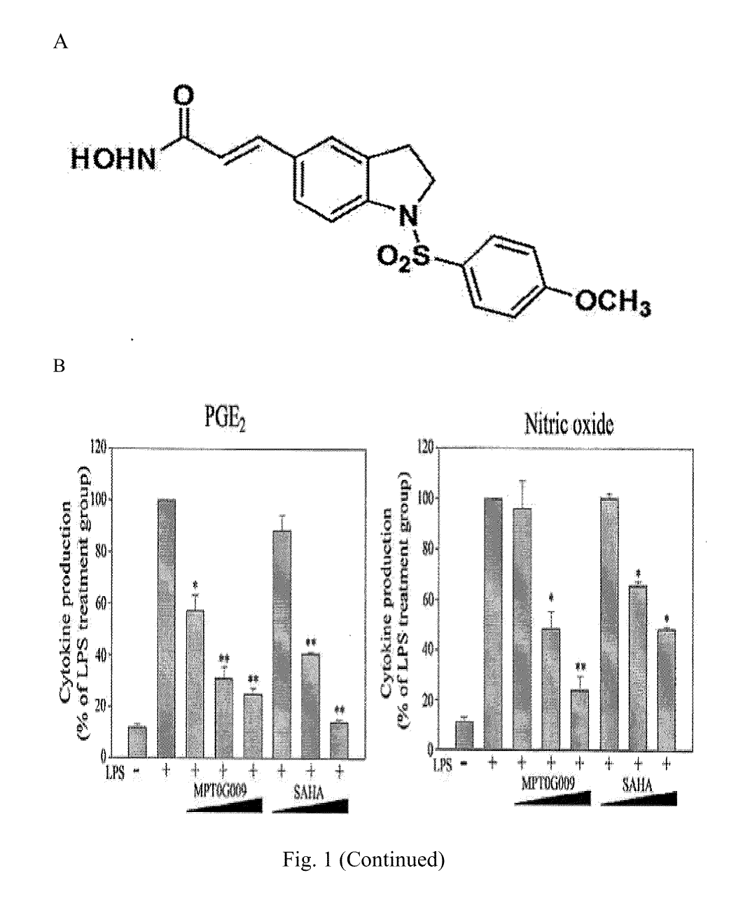

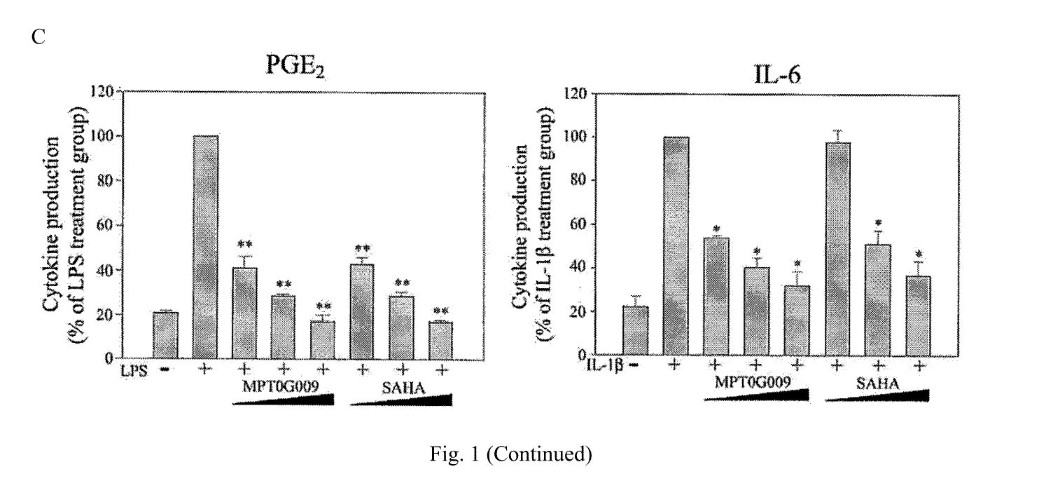

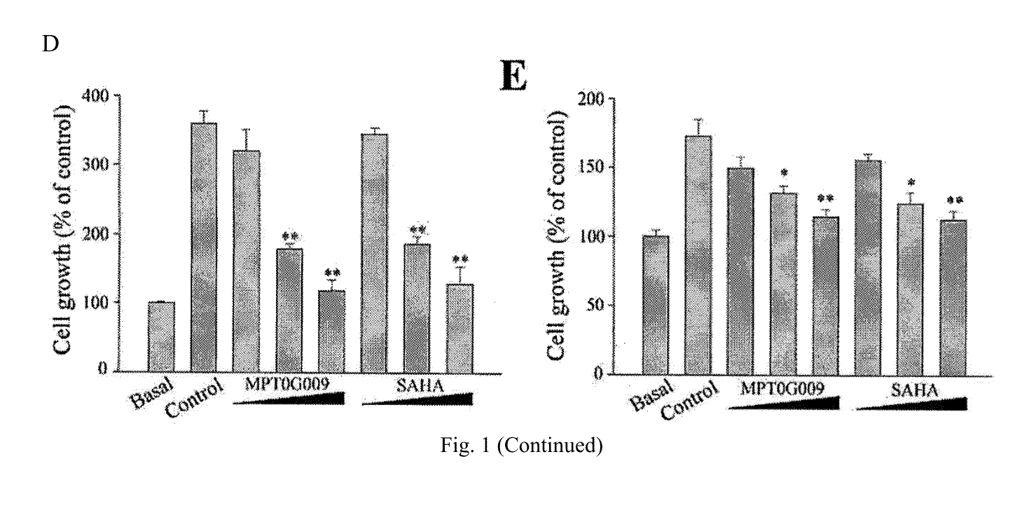

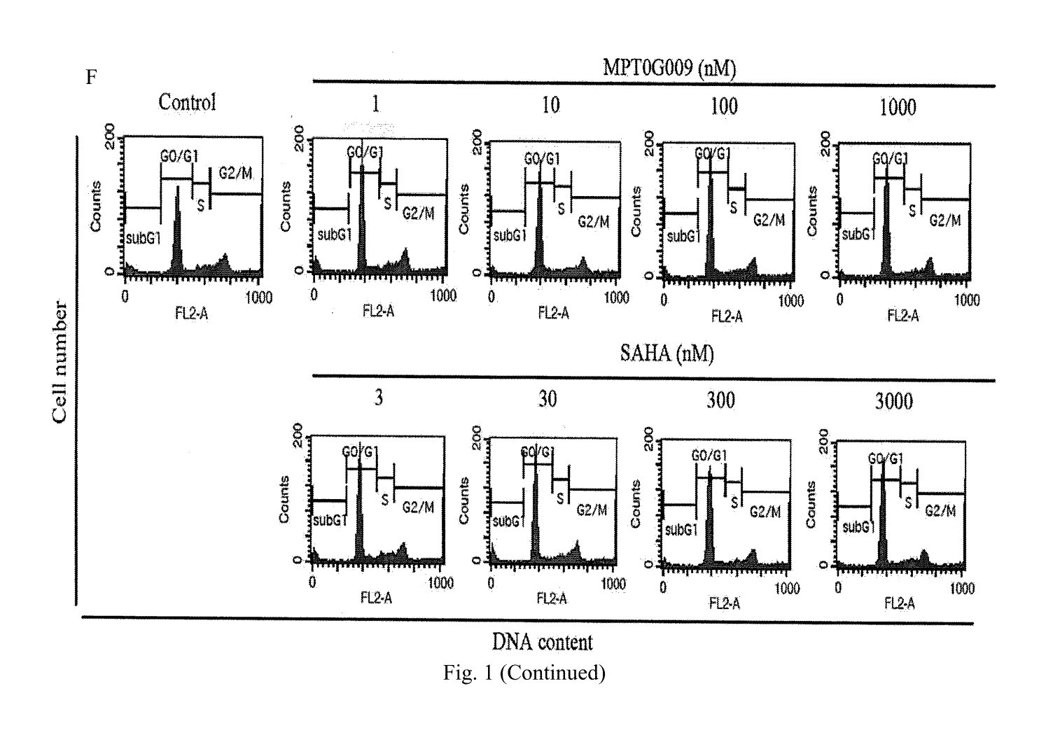

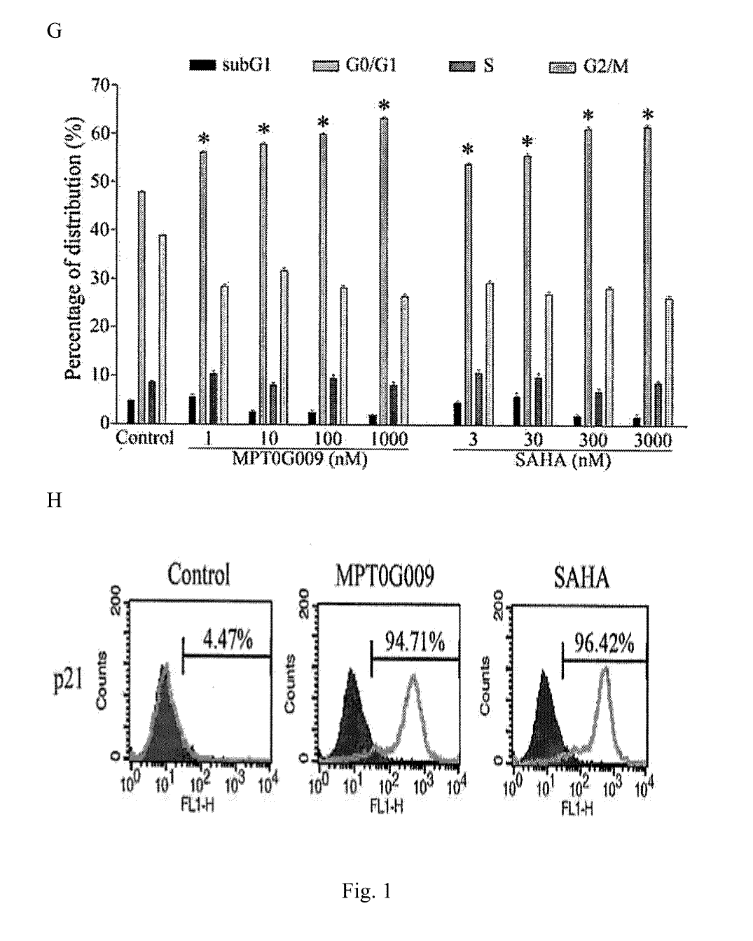

Furthermore, the anti-inflammatory effects of MPT0G009 was evaluated. Supernatants from cultures of RAW264.7 cells (FIG. 1B) and RA-FLS (FIG. 1C) were incubated with different concentrations of MPT0G009 (0, 0.1, 1, or 10 μM) or SAHA (0, 0.3, 3, or 30 μM) for 30 min before and during incubation for 24 h with lipopolysaccharide (LPS, 25 ng/mL) or IL-1β (10 ng/mL). These supernatants were then assayed for PGE₂, NO, and IL-6.

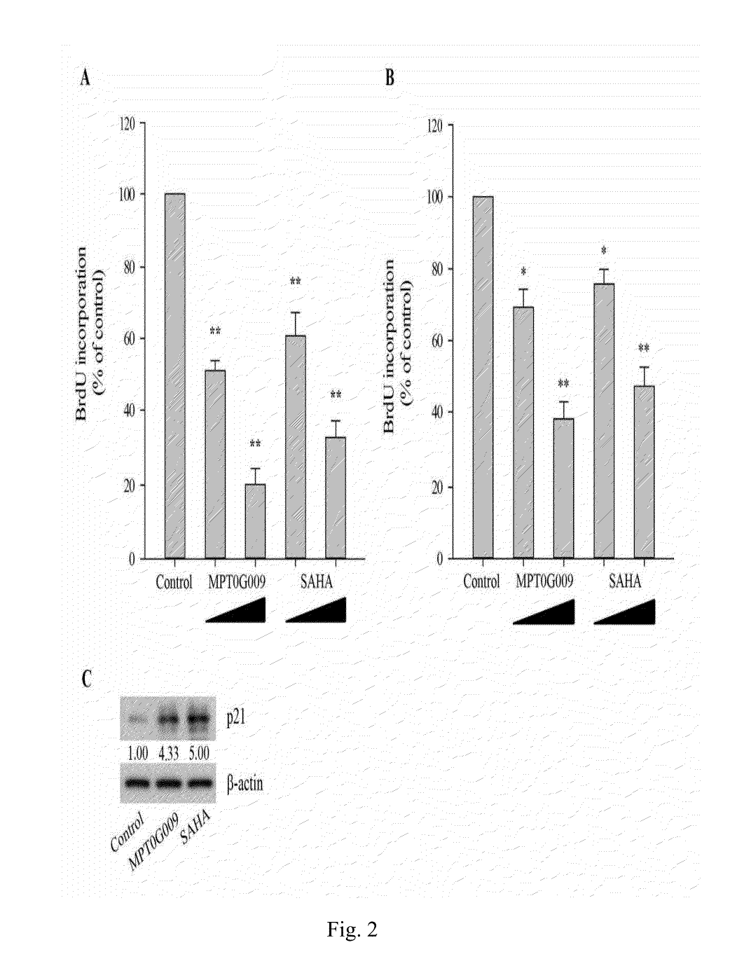

MPT0G009 and SAHA inhibited PGE₂ production by both cell types, NO production by RAW264.7 cells, and IL-6 production by RA-FLS in a concentration-dependent manner; MPT0G009 was more effective than SAHA. Because synoviocyte proliferation plays a pivotal role in RA pathogenesis, we assessed the effects of MPT0G009 and SAHA at the above mentioned concentrations on the proliferation of HIG-82 synoviocytes (FIG. 1D) or RA-FLS (FIG. 1E) after 24 or 48 h of incubation (FIGS. 2A and B). These results showed that both inhibitors had similar concentration-dependent anti-proliferative effects on both cell types.

To investigate the effects of MPT0G009 and SAHA on cell cycle progression, cellular DNA contents were determined by flow cytometry. As shown in FIGS. 1F and 1G, treating RA-FLS with MPT0G009 (1-1000 nM) or SAHA (3-3000 nM) for 24 h did not increase the subG1 peak, suggesting that these agents did not cause cellular apoptosis. However, G0/G1 phase arrest was observed after treating these cells with all concentrations of both agents. We then examined whether this was attributable to an effect on cyclin-dependent kinase inhibitors, such as p21, by incubating RA-FLS with 1 μM MPT0G009 or 3 μM SAHA for 24 h and assessed the expression of p21 by flow cytometry (FIG. 1H) or western blot (FIG. 2C). We found that treating these cells with either agent significantly increased the expression of p21, which was consistent with their effects on cell cycle distributions.

### Example 19

### Effects of Compound 9 and SAHA (1) on LPS-Induced iNOS and COX-2 Protein Expression

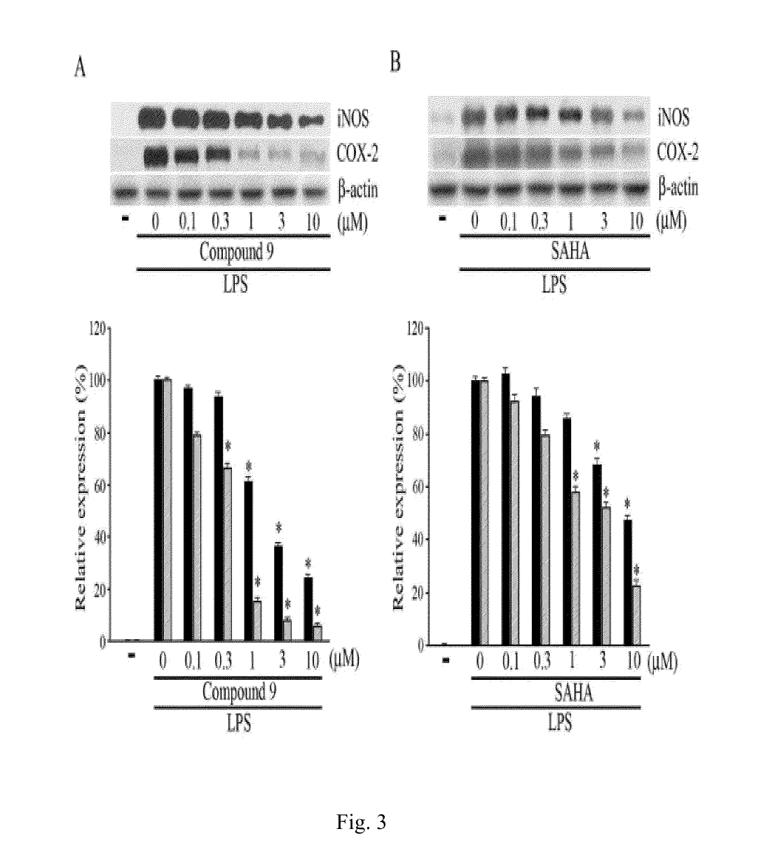

To determine whether the inhibitory effect of compound 9 and SAHA (1) on inflammatory factors NO and PGE₂ were related to the modulation of iNOS and COX-2 expression, Western blot analysis was performed. As shown in FIG. 1, compound 9 (FIG. 3A) and SAHA (FIG. 3B) significantly inhibited iNOS and COX-2 protein expression in a concentration-dependent manner.

### Example 20

### Anti-Inflammatory Effect of Compound 9 and SAHA (1) in Rats

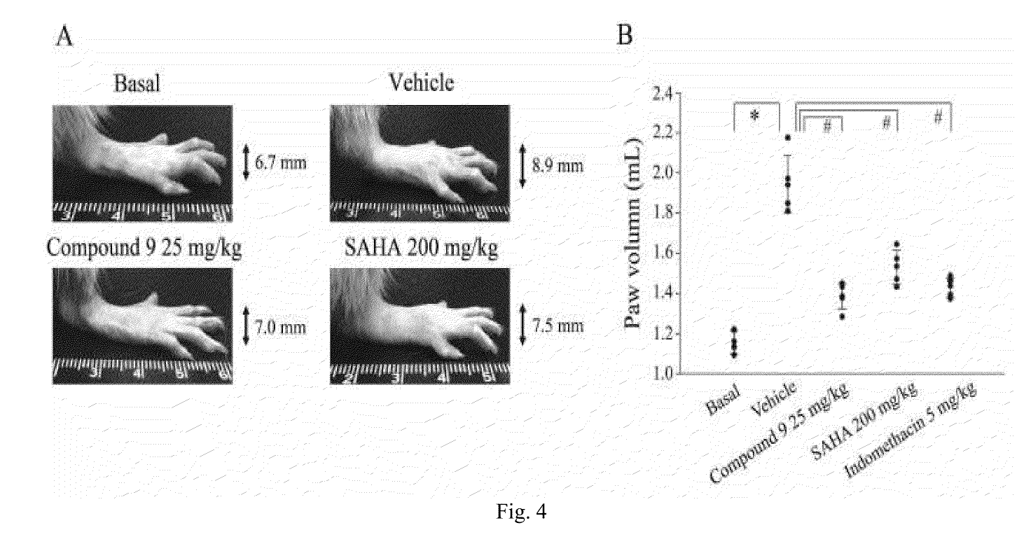

To investigate the effects of compound 9 and SAHA on inflammation models, rats were initially oral treated with compound 9 (25 mg/kg), SAHA (200 mg/kg), and Indomethacin (5 mg/kg) for 1 h and then subjected to carrageenan-induced acute inflammatory hind paw edema. The results showed that compound 9 and SAHA significantly inhibited hind paw edema (FIGS. 4A and 4B). Indomethacin, which was used as positive control, also suppressed hind paw edema. In addition, the carrageenan-induced paw thicknesses were reduced compared to control by 21.3% and 15.7% after compound 9 and SAHA treatment, respectively (FIG. 4A). These results suggest that compound 9 exhibited a potent anti-inflammatory effect. Example 21 MPT0G009 increases histone H3 acetylation in HIG-82 synovial fibroblasts and RA fibroblast-like synoviocytes.

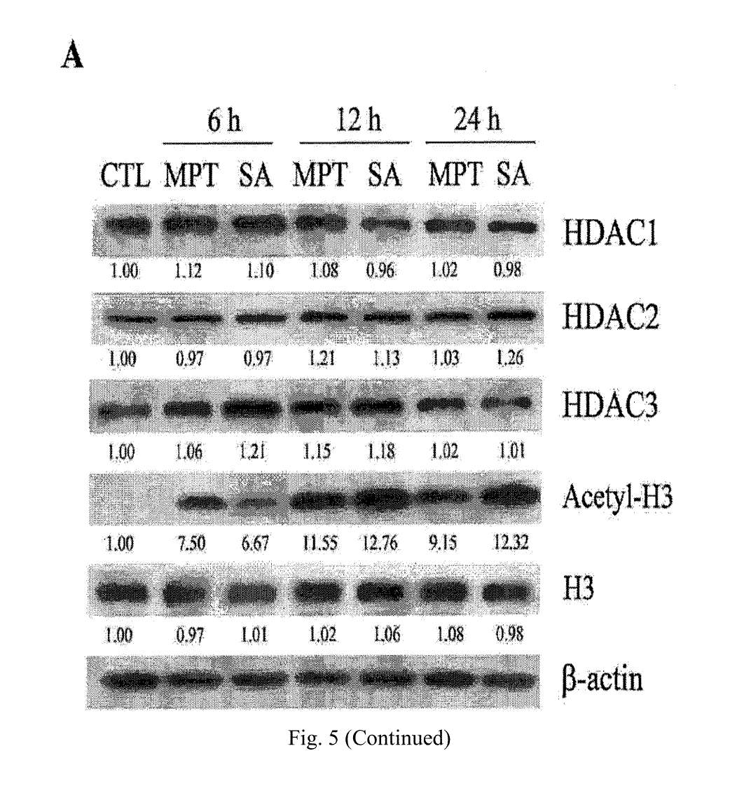

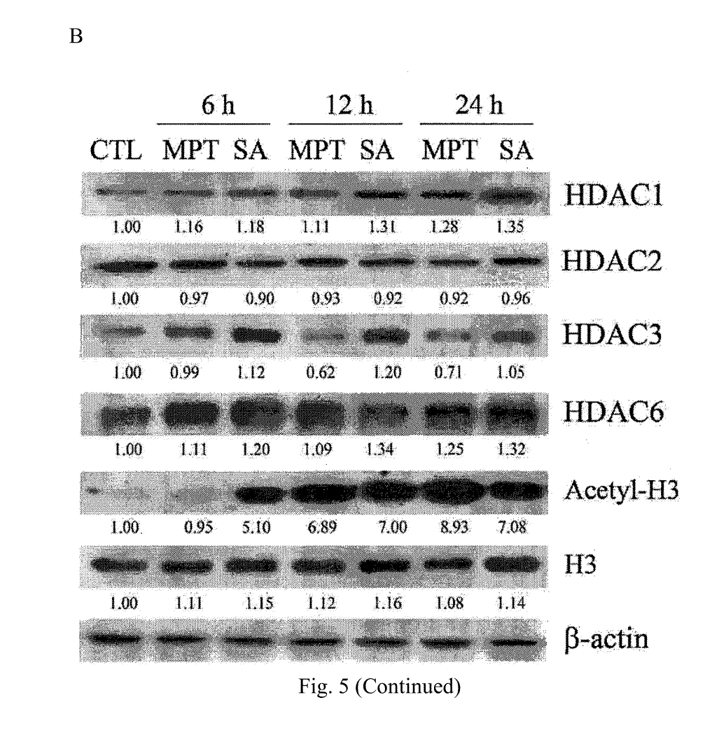

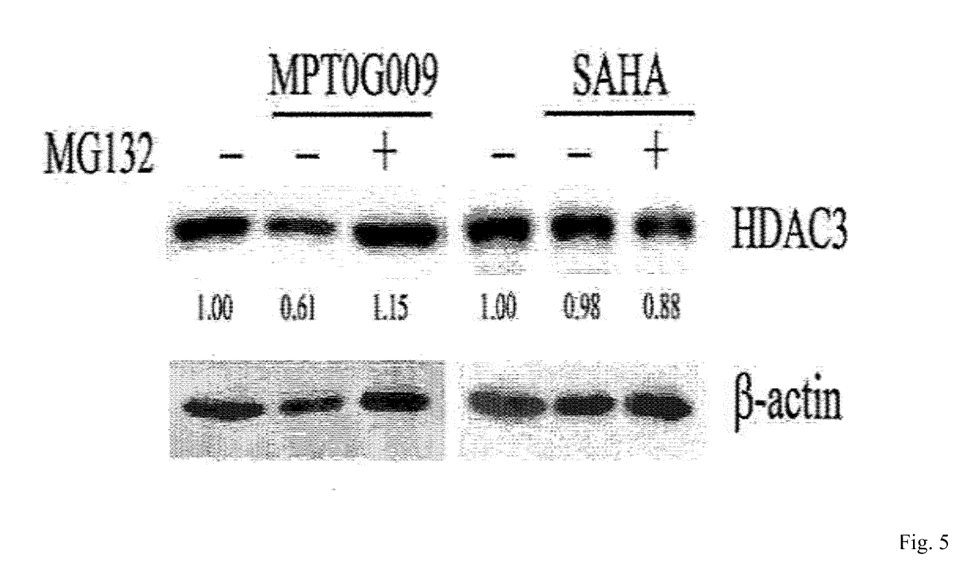

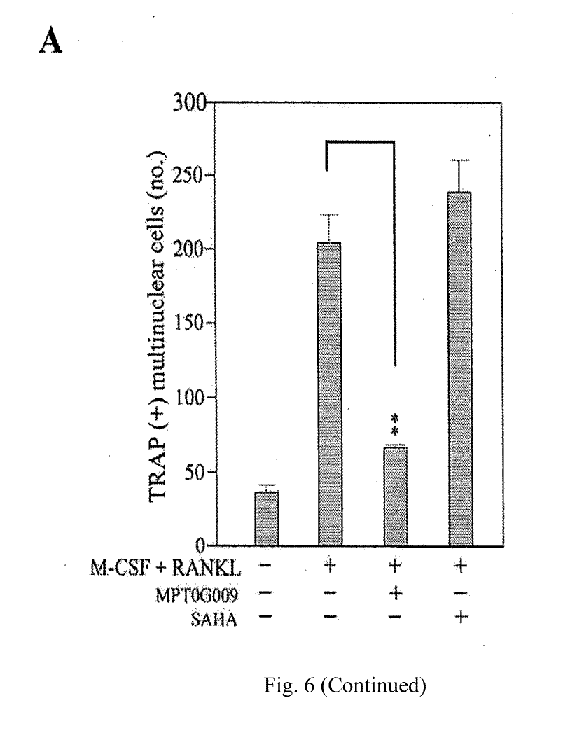

Because histone H3 is a target of HDACs, we examined whether a MPT0G009- or SAHA-induced decrease in HDAC activity resulted in changes in histone acetylation in HIG-82 synoviocytes and RA-FLS. Western blots of lysates of HIG-82 synoviocytes that were treated with 3 μM MPT0G009 (FIG. 5a) or RA-FLS that was treated with 1 μM MPT0G009 (FIG. 5b) for 6, 12, or 24 h showed that there was significant hyperacetylation of histone H3 (Acetyl-H3) starting at 6 h (HIG-82 cells) or 12 h (RA-FLS), and it was maintained for at least until 24 h as compared with those of an untreated control. SAHA treatment of both cell types (60 μM for HIG-82 synovial fibroblasts and 30 μM for RA-FLS) had a similar effect. In addition, treating RA-FLS cells, but not HIG-82 cells, with MPT0G009 for 12 or 24 h resulted in decreased levels of HDAC3 but not of the other isoforms, whereas SAHA had no effect (FIG. 5b). This was a proteasome-dependent effect because it was reduced by pretreatment with the proteasome inhibitor, MG132 (1 μM; FIG. 5c).

### Example 22

### MPT0G009 Inhibits Macrophage Colony-Stimulating Factor/Receptor Activator of NF-κB Ligand (M-CSF/RANKL)-Induced Osteoclast Formation

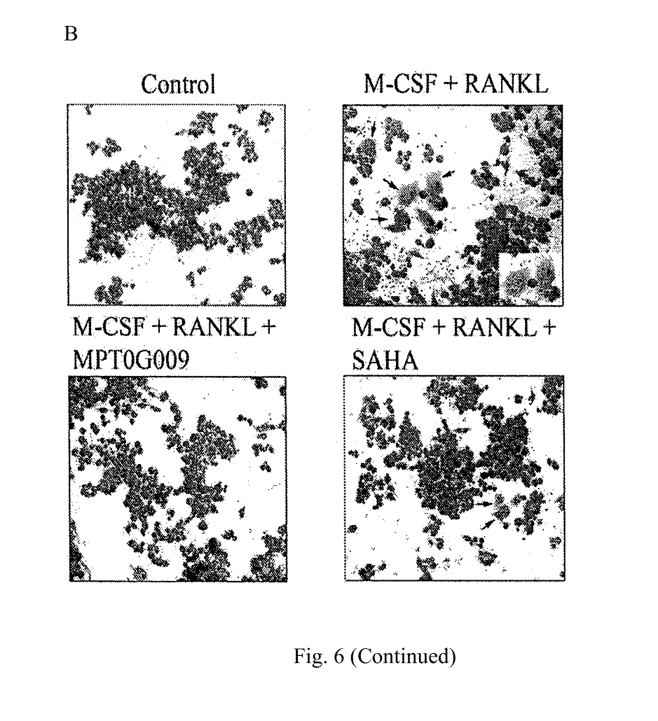

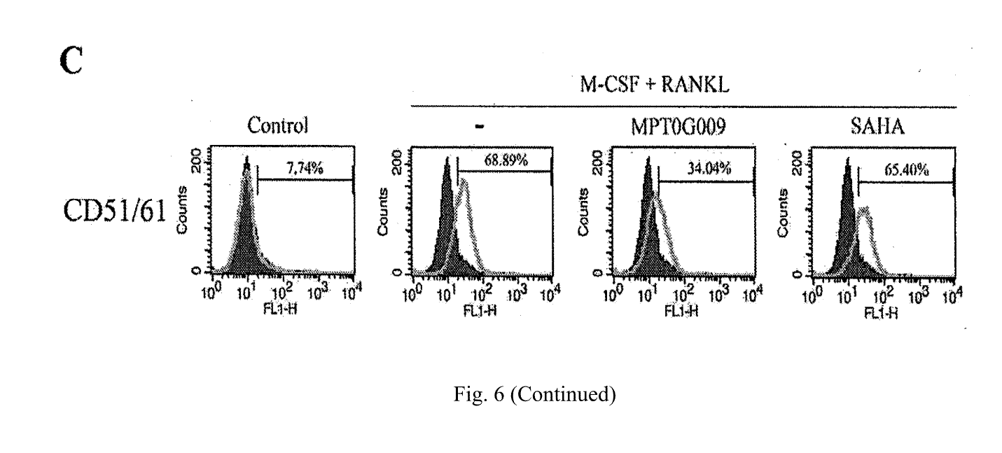

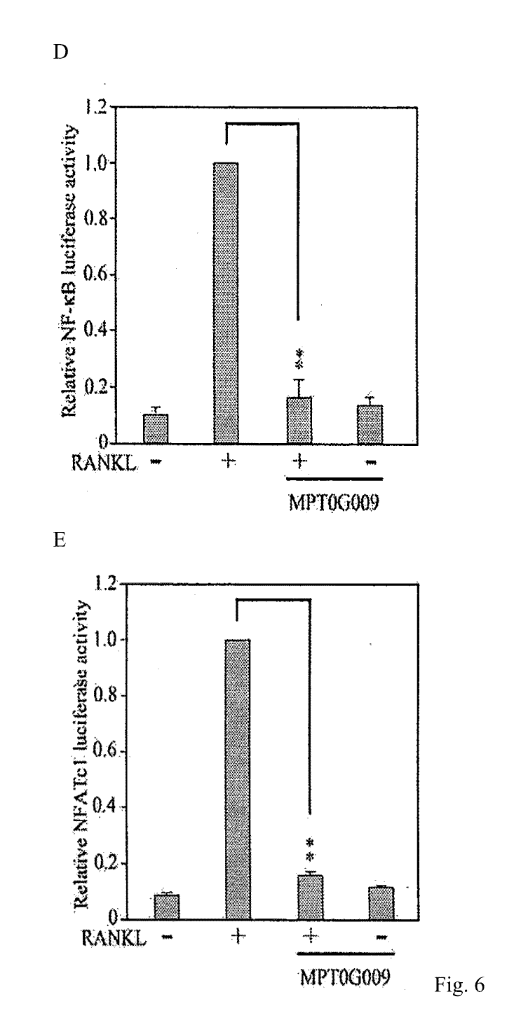

Bone destruction is one characteristic of RA pathogenesis, resulting in joint dysfunction. Differentiation of mouse macrophages osteoclast-like cells can be induced in the presence of M-CSF and RANKL, which has been used as a model to investigate osteoclast differentiation. To evaluate the effect of MPT0G009 on osteoclast formation, RAW264.7 macrophages were incubated for 30 min with or without 5 nM MPT0G009 or 50 nM SAHA (FIGS. 6a and b) before and during treatment with M-CSF/RANKL (50 ng/mL) for 5 days. Subsequently, multinucleate tartrate resistant acid phosphatase (TRAP)-positive cells were counted. In the absence of an inhibitor, M-CSF/RANKL treatment induced the formation of 205±10 TRAP-positive multinuclear osteoclast-like cells (FIGS. 6a and b), and 68.9±3.7% of these cells were positive for the osteoclast-specific marker CD51/61 (FIG. 6c). The concentrations of MPT0G009 that were used (5 nM) significantly inhibited the formation of M-CSF/RANKL-induced TRAP-positive cells (FIG. 6a), and significantly inhibited the expression of the osteoclast-specific marker (FIG. 6c). In contrast, SAHA treatment had no effect, even at 50 nM (FIGS. 6a-c).

We also assessed the effect of MTPOG009 on the DNA-binding activity of NF-kB and NFATc1, two pivotal transcriptional factors involved in RANKL-induced pathways for promoting osteoclast differentiation. When RAW264.7 cells that had been transiently transfected with reporter plasmids were treated with 5 nM MPT0G009 for 30 min before and during 24-h stimulation with RANKL, MPT0G009 inhibited RANKL-induced NF-kB (FIG. 6d) and NFATc1 (FIG. 6e) luciferase activity. These results showed that MPT0G009 could inhibit M-CSF/RANKL-induced osteoclast differentiation and signals.

### Example 23

### MPT0G009's Inhibitory Effects on Cytokine Production and Osteoclast Differentiation are Reduced by the Overexpression of HDAC1 and HDAC6

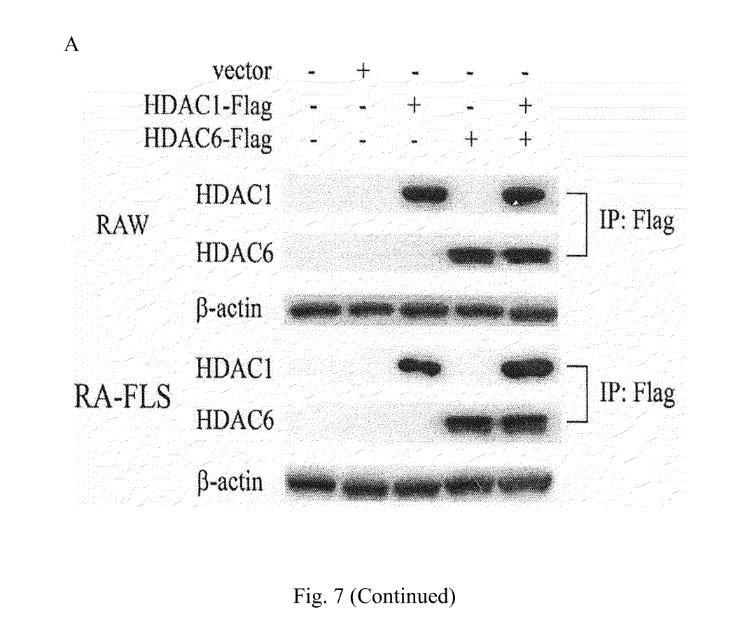

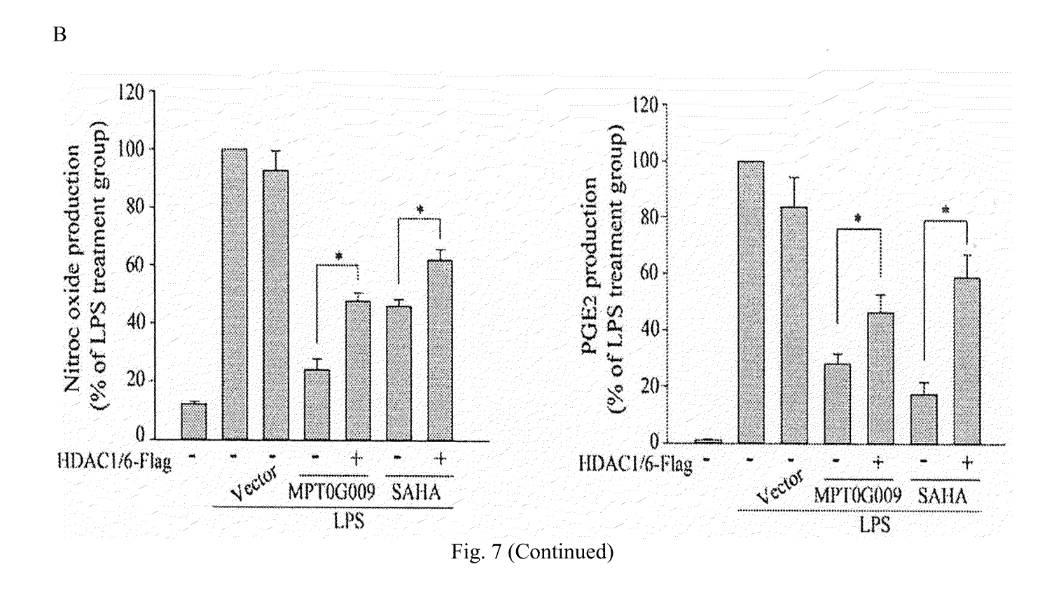

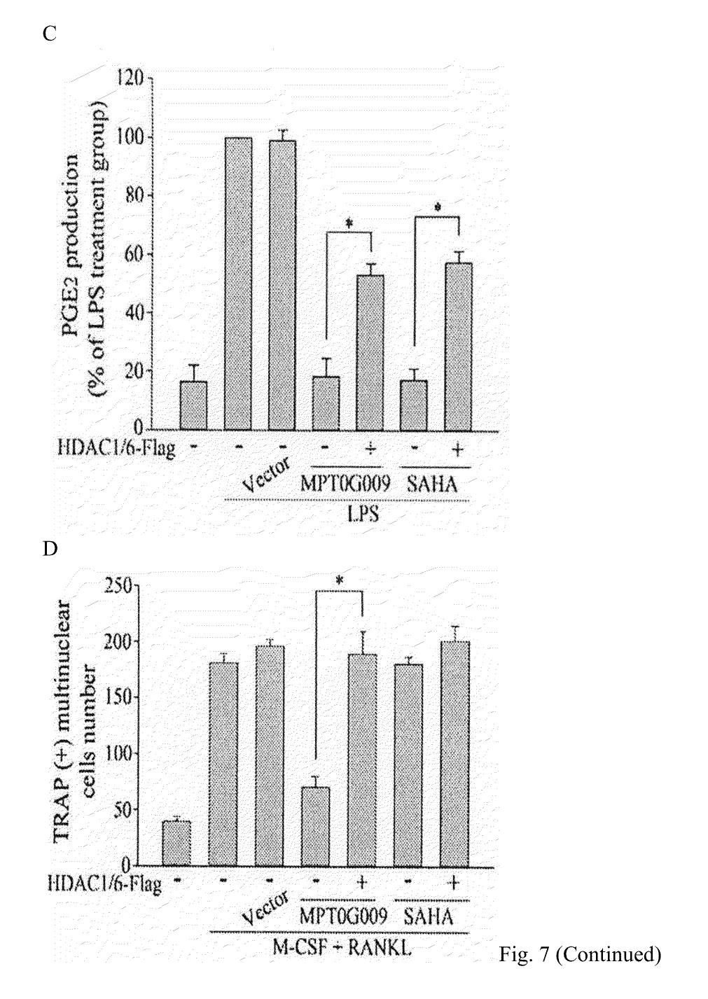

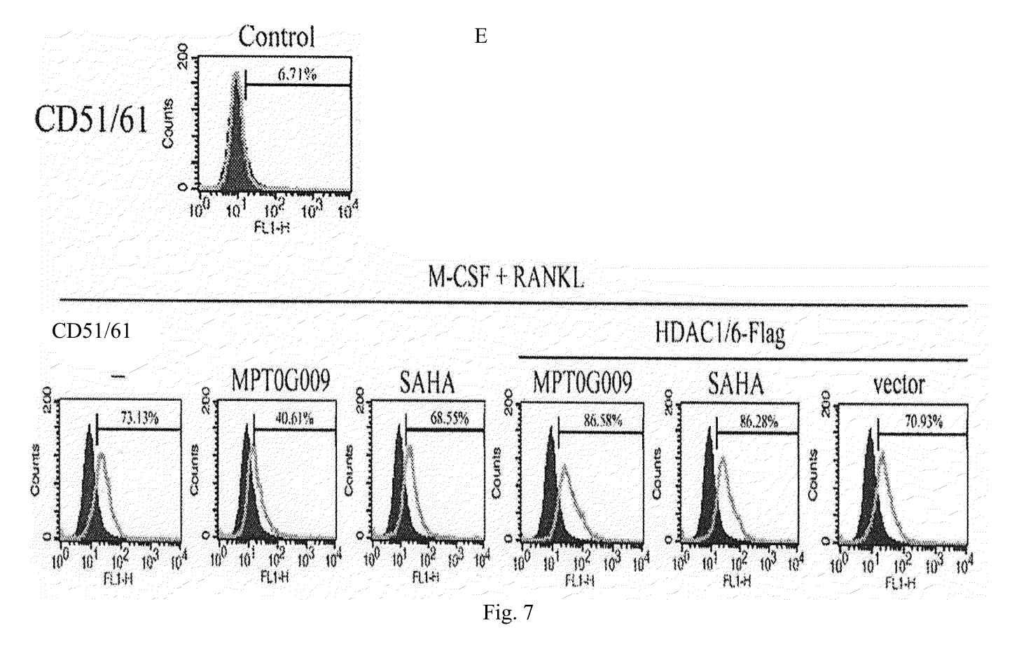

Next, we examined whether MPT0G009 inhibited cytokine release and osteoclast formation by inhibiting HDAC activity. As shown in FIG. 7a, RAW264.7 macrophages and RA-FLS that were transfected with HDAC1- and/or HDAC6-encoding plasmid(s) expressed the expected isoforms(s). Empty vector-transfected or HDAC1- and HDAC6-coexpressing RAW264.7 macrophages (FIG. 7b) or RA-FLS (FIG. 7c) were incubated for 30 min with or without 10 μM MPT0G009 or 30 μM SAHA. Then LPS (25 ng/mL) was added for 24 h, and nitrite and PGE₂ levels were measured in culture supernatants. These results showed that overexpression of HDAC significantly reduced the inhibitory effects of MPT0G009 or SAHA on LPS-mediated NO or PGE₂ release (FIGS. 7b and c) and that of MPT0G009 on M-CSF/RANKL-induced osteoclast differentiation (FIG. 7d) as well as the expression of the osteoclast-specific marker, CD51/61 (FIG. 7e). Thus, MPT0G009 apparently inhibited cytokine release and osteoclast differentiation.

### Example 24

### MPT0G009 Inhibits the Development of Arthritis in an Adjuvant-Induced Arthritis (AIA) Model

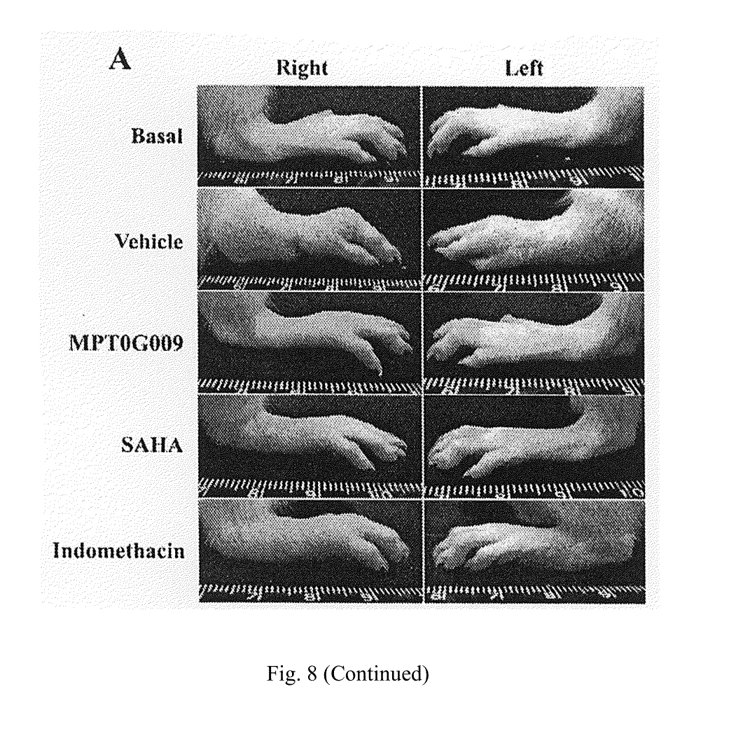

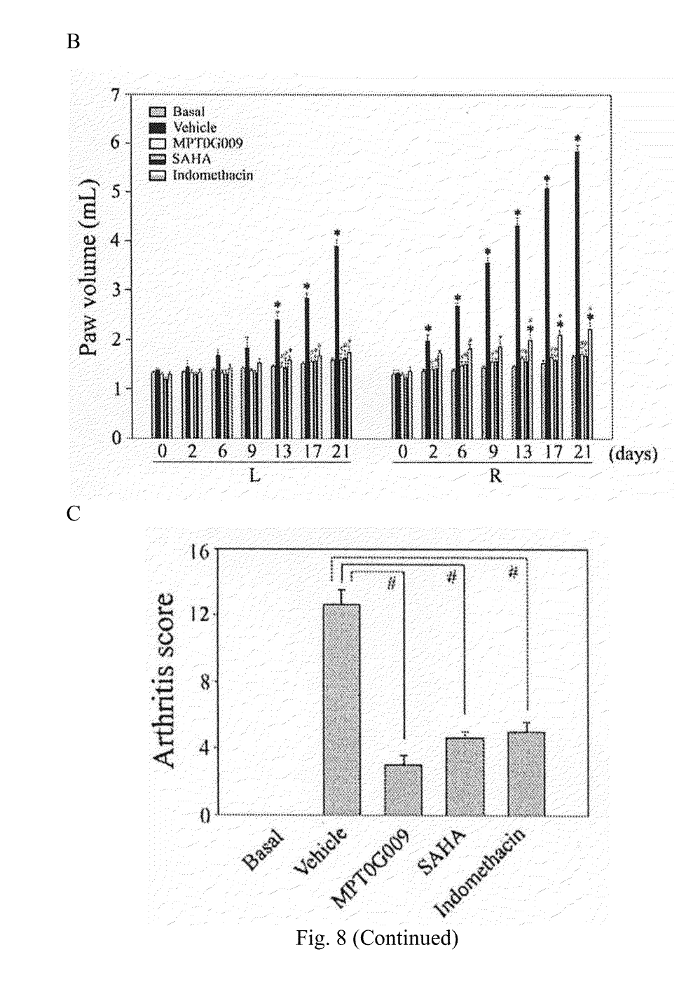

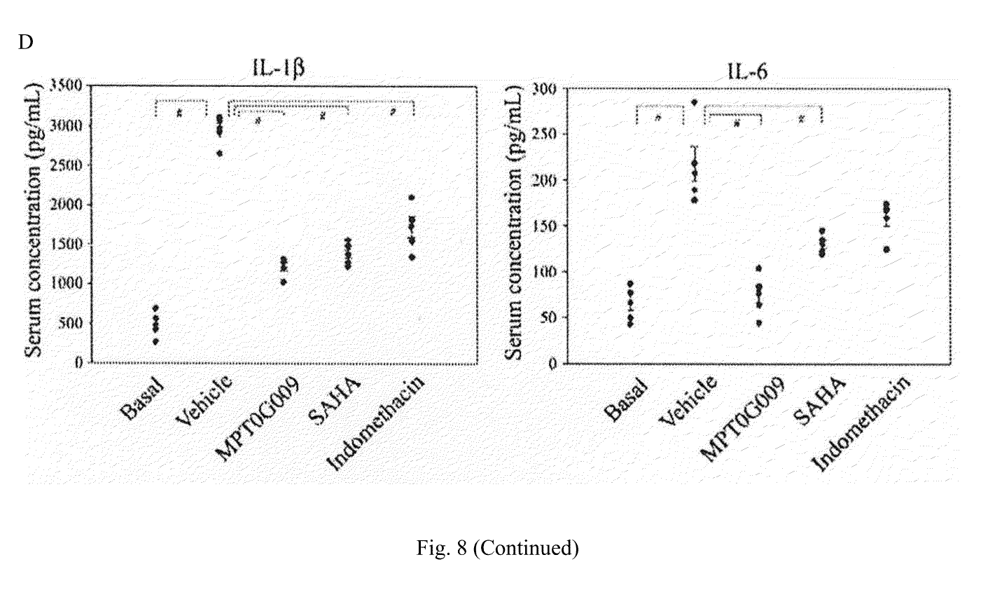

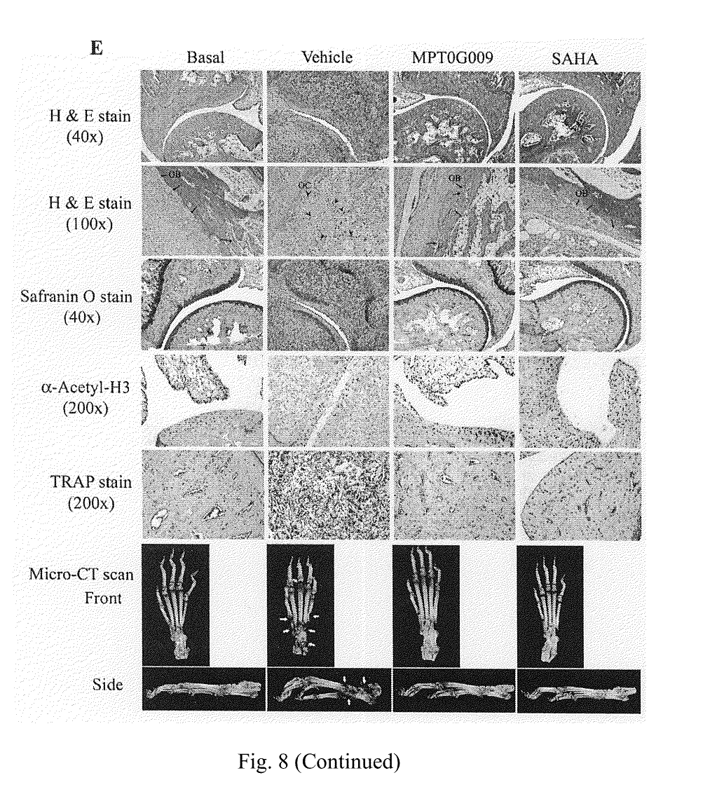

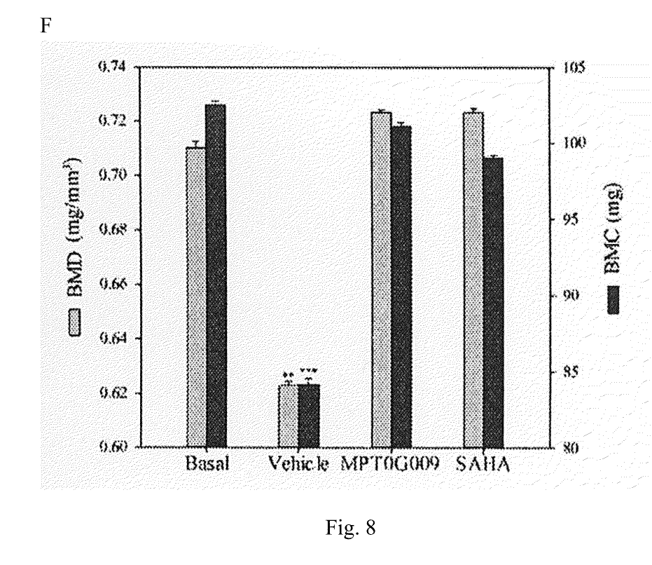

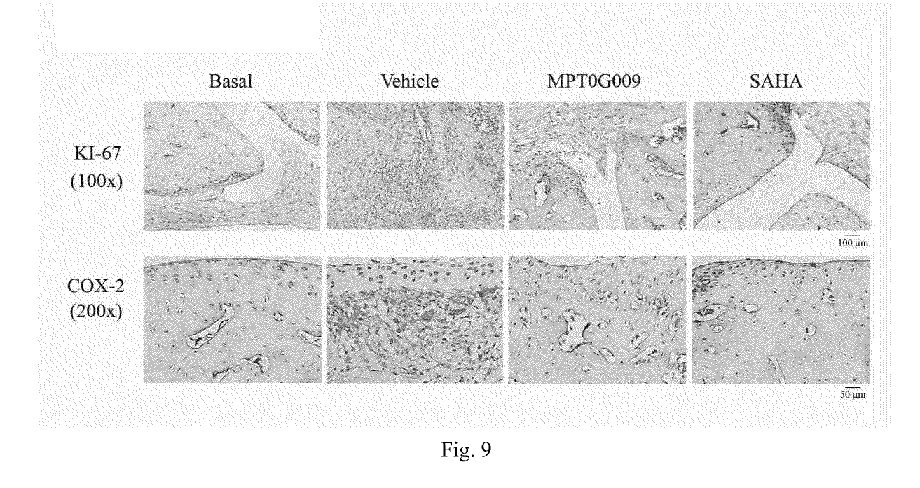

Next, we evaluated the in vivo anti-arthritic effects of MPT0G009 in a rat AIA model. As shown in FIG. 5, compared with the vehicle-treated group, the group treated with 25 mg/kg of MPT0G009 daily from days 2 to 21 had significant reductions in paw swelling (FIG. 8a), paw volume (FIG. 8b), and arthritis scores (FIG. 8c). Similar results were found with SAHA (200 mg/kg) and NSAIDs (indomethacin; 1 mg/kg). MPT0G009 treatment resulted in significant decreases in the serum levels of IL-1β and IL-6, as did SAHA and indomethacin treatments (FIG. 8d). Furthermore, as shown in FIG. 8e, safranin O staining of rat ankle joints showed that MPT0G009 treatment markedly reduced cartilage degradation, and hematoxylin and eosin staining showed that MPT0G009 treatment significantly reduced leukocyte infiltration, synovitis and apparently ameliorated the decrease of osteoblasts. Immunohistochemical staining with an anti-acetyl-histone H3 antibody showed that the MPT0G009-treated group had increased levels of acetyl-histone H3, and TRAP stain demonstrated that MPT0G009 treatment significantly decreased the formation of osteoclasts. The inhibition of synoviocytes proliferation and inflammation by MPT0G009 treatment was also observed (FIG. 9). In addition, micro-computed tomography scans showed that MPT0G009 treatment ameliorated bone destruction

(FIG. 8e) and prevented the decrease of bone mineral density (BMD) and bone mineral content (BMC) (FIG. 8f). In these experiments, 25 mg/kg of MPT0G009 had effects similar to 200 mg/kg of SAHA and showed that it had very potent anti-arthritic effects in vivo.

### Example 25

### In Vivo Rat Pharmacokinetic Profiling and Maximum Tolerated Dose of MPT0G009

The pharmokinetic parameters of MPT0G009 in rats after single-dose intravenous (i.v.) and oral administration are summarized in Table 2. After i.v. administration, the half-life of MPT0G009 was 6.74 h, and systemic exposure and clearance were 665 ngh/mL and 5.12 L/h/kg, respectively. After oral administration, MPT0G009 showed Tₘₐₓ=3.43 h, T_{1/2}=9.53 h, and bioavailability (F)=13.0% (Table 3). Table 4 shows the maximum tolerated dose data for MPT0G009 in CD-1 mice with a daily×7 schedule. No significant adverse effects were observed within three weeks in a study of mice when MPT0G009 was administrated at a dosage of up to 1000 mg/kg/day.

TABLE 3
Intravenous and oral pharmacokinetic parameters
of MPT0G009 and SAHA in rats

| | MPT0G009 | | SAHA³⁰ | |
|---|---|---|---|---|
| | iv | po | iv | po |
| Parameters | 2 mg/kg | 20 mg/kg | 2 mg/kg | 5 mg/kg |
| CL (L/h/kg) | 5.12 | — | 8.04 | — |
| V_{d} (L/kg) | 25.21 | — | 3.92 | — |
| T_{1/2} (h) | 6.74 | 9.53 | 0.64 | 1.13 |
| AUC_{(0-inf)} (ng h/mL) | 665 | 1265 | 278 | 49.40 |
| Tₘₐₓ (h) | — | 3.43 | — | 0.17 |
| Cₘₐₓ (ng/mL) | — | 221 | — | 83.90 |
| F (%) | — | 13.0 | — | 6.9 |

TABLE 4
The maximum tolerated dose (MTD) of MPT0G009

| | Dose | | | Change of body weight (%) | | | |
|---|---|---|---|---|---|---|---|
| Comound | (mg/kg) | Schedule | Mouse No. | Day 7 | Day 14 | Day 21 | Lethality (%) |
| MPT0G009 | 250 | po, qd × 7 | 6 | +8.7 ± 0.5 | +17.0 ± 0.4 | +22.6 ± 0.6 | 0 |
| | 500 | po, qd × 7 | 6 | +1.6 ± 0.3 | +16.5 ± 0.6 | +24.0 ± 0.9 | 0 |
| | 1000 | po, qd × 7 | 6 | +4.4 ± 0.6 | +12.0 ± 0.7 | +15.3 ± 0.8 | 0 |

Data in the column of Change of body weight represent mean ± SEM.

### Example 26

### MPT0E028 and MPT0G009 Inhibit Pro-Fibrogenic Mediators-Induced Fibrotic Protein, CTGF and Collagen I, Production

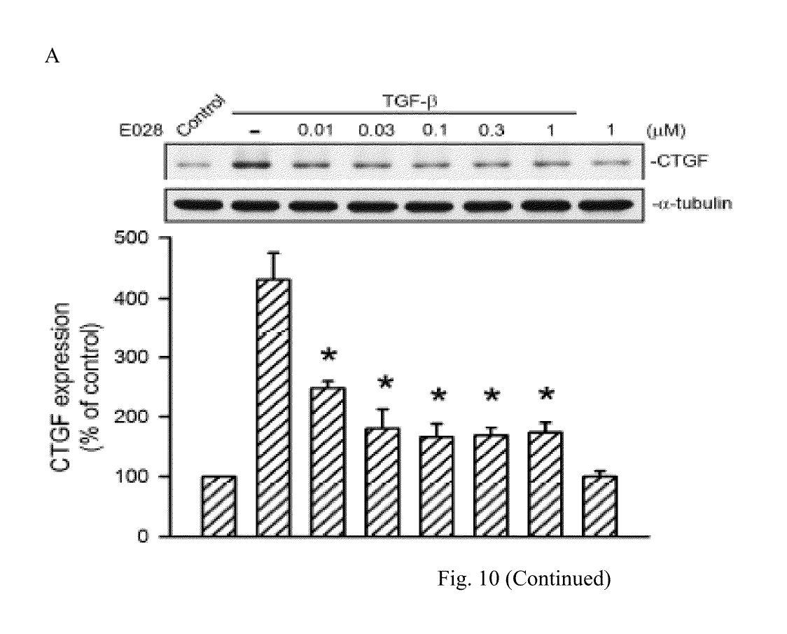

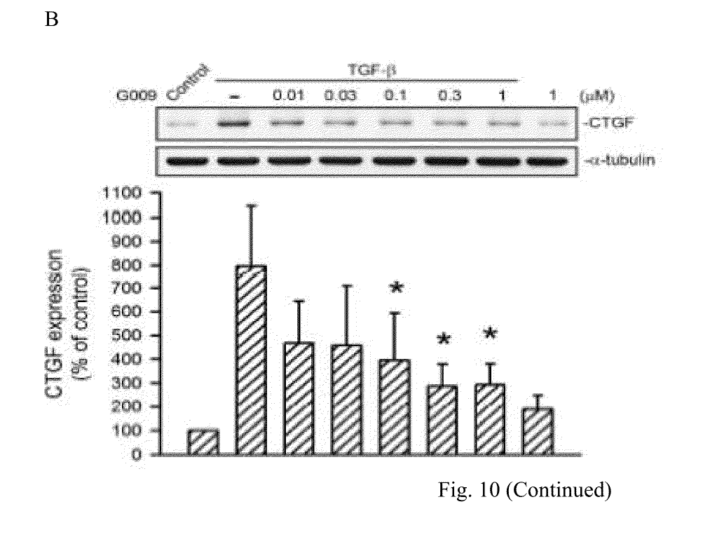

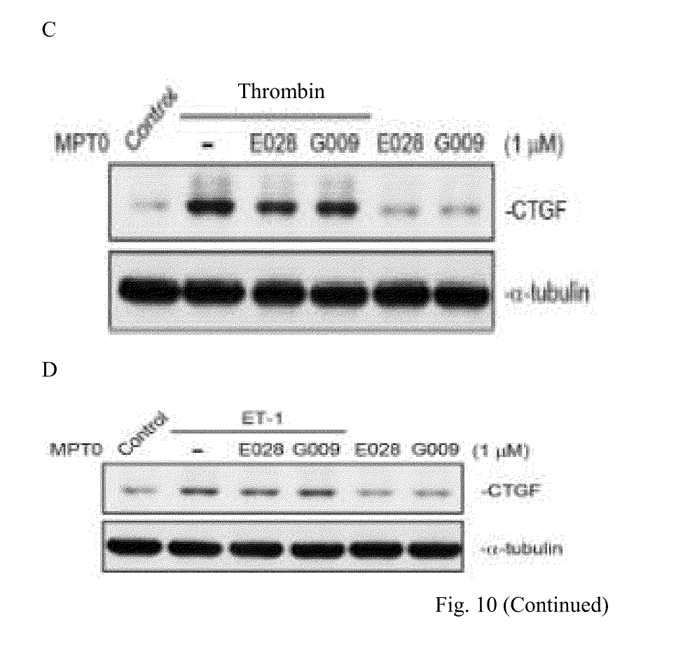

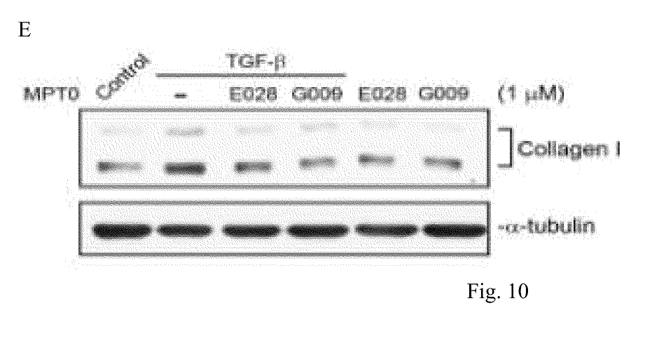

To determine whether the inhibitory effects of MPT0E028 and MPT0G009 on pro-fibrogenic mediators (TGF-β, thrombin, and ET-1) were related to the modulation of CTGF and Collagen I production, Western blot analysis was performed. First, WI-38 lung fibroblasts were incubated with different concentrations of MPT0E028 (0.01, 0.03, 0.1, 0.3, or 1 μM) (FIG. 10A) or MPT0G009 (0.01, 0.03, 0.1, 0.3, or 1 μM) (FIG. 10B) for 30 min before and during incubation for 2 h with TGF-β (10 ng/mL). Both MPT0E028 and MPT0G009 significantly inhibited TGF-β-induced CTGF production from WI-38 lung fibroblasts in a concentration-dependent manner. Furthermore, we examined the effects of MPT0E028 and MPT0G009 on other pro-fibrogenic mediators. As shown in FIGS. 10C and 10D, WI-38 lung fibroblasts were incubated with 1 μM MPT0E028 and MPT0G009 for 30 min and following another incubation for 2 h with 1 U/ml thrombin (FIG. 10C) and 10 nM ET-1 (FIG. 10D). MPT0E028 and MPT0G009 also inhibited thrombin- and ET-1-induced CTGF expression form WI-38 lung fibroblasts. In addition, as shown in FIG. 10E, the collagen I production from WI-38 lung fibroblasts stimulated by 10 ng/mL TGF-β for 24 h was inhibited with 1 μM MPT0E028 and MPT0G009 pretreatment. These results showed that MPT0E028 and MPT0G009 apparently inhibited pro-fibrogenic mediators-induced CTGF and collagen I production by inhibiting HDAC activity.

What is claimed is:

1. A method for inhibiting cytokine release from a cell or a subject, comprising administering an effective amount of the compound having formula (I) or a pharmaceutically acceptable salt, prodrug or solvate thereof to the cell or the subject: 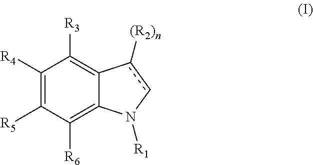
wherein [symbol] is a single bond or a double bond;
R₁ is SO₂Rₐ, wherein Rₐ is aryl unsubstituted or substituted by 1 to 3 substituent selected from the group consisting of: —OC₁₋₁₀alkyl, halogen, —NO₂, —NH₂, —OH, —C₁₋₆alkyl, —C₂₋₁₀alkenyl, —C₂₋₁₀alkynyl, —C₃₋₁₀cycloalkyl, —C₁₋₁₀cycloalkenyl, 6 to 10 membered aryl or 6 to 10 membered heteroaryl;
R₂, R₃, R₅ and R₆ are each independently H, —OC₁₋₁₀alkyl, halogen, —NO₂, —NH₂, —OH, —C₁₋₁₀alkyl, —C₂₋₁₀alkenyl or —C₂₋₁₀alkynyl; and
R₄ is H, C₁₋₁₀alkyl, C₂₋₁₀alkenyl, C₂₋₁₀alkynyl, aryl, 5 to 14 membered heteroaryl, C₃₋₁₀cycloalkyl, C₅₋₁₀cycloalkenyl, C₃₋₁₄heterocycloC₁₋₁₀alkyl, C₅₋₁₄heterocyclo C₂₋₁₀alkenyl, halo, cyano, nitro, OR_{b}, SR_{b}, S(O)R_{b}, CH═CH—C(O)NR_{c}R_{d}, NHC(O)—CH═CH—C(O)R_{b}, NHC(O)—CH═CH—C(O)NRcRd, SO₂NRcRd, OC(O)R_{b}, C(O)NR_{c}R_{d}, NRcRd, NHC(O)R_{b}, NHC(O)NR_{c}R_{d}, or NHC(S)Rc, in which each of R_{b}, R_{c}, and R_{d}, independently, is H, hydroxy, C₁₋₁₀alkoxy, C₆₋₁₀aryloxy, C₅₋₁₄heteroaryloxy, C₁₋₁₀alkyl, C₂₋₁₀alkenyl, C₂₋₁₀alkynyl, C₆₋₁₀aryl, C₅₋₁₄heteroaryl, C₃₋₁₀cycloalkyl, C₅₋₁₀cycloalkenyl, C₃₋₁₄heterocycloC₁₋₆alkyl, or C₅₋₁₄heterocycloC₂₋₁₀alkenyl.

2. The method of claim 1, wherein aryl is 6 to 10 membered aryl; C₁₋₁₀alkyl is C₁₋₄alkyl or C₁₋₆alkyl; C₂₋₁₀alkenyl is C₂₋₆alkenyl; or C₂₋₁₀alkynyl is C₂₋₆alkynyl.

3. The method of claim 1, wherein Rₐ is 6 to 10 membered aryl unsubstituted or substituted by 1 to 3 substituent selected from the group consisting of: —OC₁₋₆alkyl, halogen, —NO₂, —NH₂, or —OH.

4. The method of claim 1, wherein Rₐ is phenyl.

5. The method of claim 1, wherein R₄ is CH═CH—C(O)NR_{c}R_{d}, NHC(O)—CH═CH—C(O)R_{b}, NHC(O)—CH═CH—C(O)NRcRd, NHC(O)R_{b}, NHC(O)NR_{c}R_{d}, or NHC(S)Rc.

6. The method of claim 1, wherein R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or —OH. Preferably, R₄ is CH═CH—C(O)NR_{c}R_{d}, and Rₐ is phenyl or naphthyl.

7. The method of claim 1, wherein [symbol] is a double bond, R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or —OH.

8. The method of claim 7, wherein R₄ is CH═CH—C(O)NR_{c}R_{d}, and Rₐ is phenyl or naphthyl.

9. The method of claim 1, wherein [symbol] is a single bond, R₄ is CH═CH—C(O)NR_{c}R_{d}, and Rₐ is a 6 to 10 membered aryl unsubstituted or substituted by 1 to 3 substituent selected from the group consisting of: —OC₁₋₆alkyl, halogen, —NO₂, —NH₂, or —OH.

10. The method of claim 9, wherein Rₐ is phenyl or naphthyl unsubstituted or substituted by 1 to 3 substituent selected from the group consisting of: —OC₁₋₆alkyl, halogen, —NO₂, —NH₂, or —OH.

11. The method of claim 9, wherein Rₐ is phenyl or naphthyl unsubstituted or substituted by 1 to 2 substituent selected from the group consisting of: —OCH₃, halogen, —NO₂, —NH₂, or —OH.

12. The method of claim 1, wherein Ra is phenyl substituted by one to three, same or different, —OCH3, halogen, NO₂ or NH₂.

13. The method of claim 1, wherein the compound is one of the following compounds:

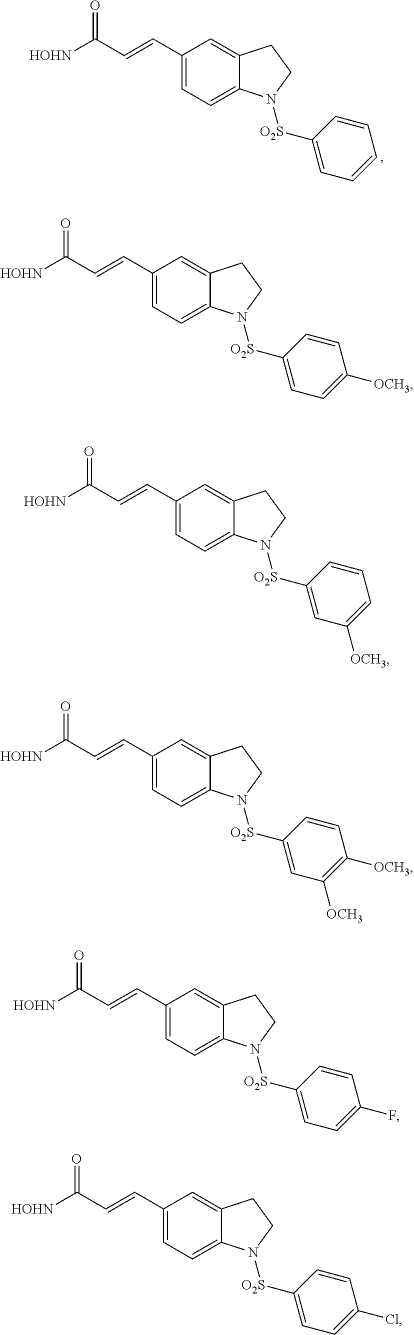

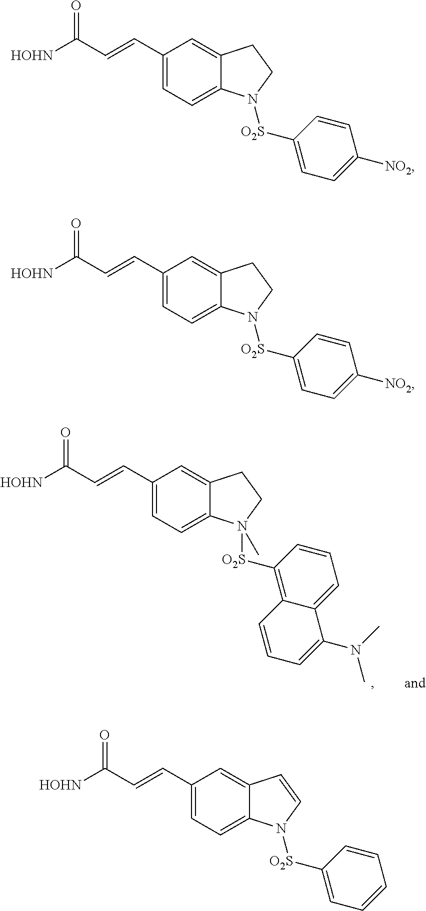

14. The method of claim 1, wherein the compound has the following structure:

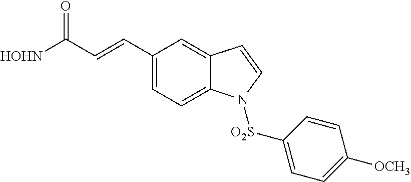

15. The method of claim 1, wherein the inhibition of cytokine release is associated with an inflammatory disease.

16. The method of claim 1, wherein the inhibition of cytokine release is associated with a chronic inflammation disease.

17. The method of claim 15, wherein inflammatory disease include arthritis (such as rheumatoid arthritis, juvenile rheumatoid arthritis, osteoarthritis and psoriatic arthritis), synovitis, vasculitis, conditions associated with inflammation of the bowel (such as Crohn's disease, ulcerative colitis, inflammatory bowel disease and irritable bowel syndrome), atherosclerosis, multiple sclerosis, Alzheimer's disease, vascular dementia, pulmonary inflammatory diseases (such as asthma, chronic obstructive pulmonary disease and acute respiratory distress syndrome), fibrotic diseases (including idiopathic pulmonary fibrosis, cardiac fibrosis and systemic sclerosis (scleroderma)), inflammatory diseases of the skin (such as contact dermatitis, atopic dermatitis and psoriasis), systemic inflammatory response syndrome, sepsis, inflammatory and/or an autoimmune disorder (for example, autoimmune conditions of the liver (such as autoimmune hepatitis, primary biliary cirrhosis, alcoholic liver disease, sclerosing cholangitis, and autoimmune cholangitis), and/or the complications thereof.

18. The method of claim 17, wherein the arthritis is osteoarthritis, rheumatoid arthritis, juvenile idiopathic arthritis, spondyloarthropathies like ankylosing spondylitis, reactive arthritis (Reiter's syndrome), psoriatic arthritis, enteropathic arthritis associated with inflammatory bowel disease, Whipple disease and Behcet disease, septic arthritis, gout (also known as gouty arthritis, crystal synovitis, metabolic arthritis), pseudogout (calcium pyrophosphate deposition disease) or Still's disease.

19. The method of claim 17, wherein the fibrotic disease is pulmonary fibrosis, liver fibrosis or renal fibrosis.

20. The method of claim 1, wherein the compound is in combination with one or more pharmaceutically acceptable excipients.

21. The method of claim 1, wherein the compound is administered in the amount of 25-250 mg/kg.

22. The method of claim 1, wherein the compound is administered orally.

23. The method of claim 1, wherein the compound is administered parenteral.

24. A method for inhibiting HDACs 1, 2, 3, and 8 in a cell or a subject, comprising administering an effective amount of the compound having formula (I) or a pharmaceutically acceptable salt, prodrug or solvate thereof to the cell or subject.
